(19) United States
(12) Patent Application Publication
(10) Pub. No.: US 2023/0399392 A1
(43) Pub. Date: Dec. 14, 2023

(54) COMPOSITION FOR PREVENTING OR TREATING PULMONARY FIBROSIS DISEASE

(71) Applicant: FNCT BIOTECH, INC., Seoul (KR)

(72) Inventors: Su Jae LEE, Seoul (JP); In Yeong YUN, Seoul (KR); Jae Hyeok KANG, Seoul (KR)

(73) Assignee: FNCT BIOTECH, INC., Seoul (KR)

(21) Appl. No.: 18/026,050

(22) PCT Filed: Sep. 14, 2021
(86) PCT No.: PCT/KR2021/012532

§ 371 Date: Mar. 13, 2023

(30) Foreign Application Priority Data
Sep. 14, 2020 (KR) 10-2020-0117803

(51) Int. Cl. C07K 16/24; A61P 11/00

(52) U.S. Cl. CPC C07K 16/243; A61P 11/00

(57) ABSTRACT

A novel use of granulocyte colony-stimulating factor, also known as colony-stimulating factor 3 (CSF3) is disclosed. An anticancer adjuvant containing an inhibitor of CSF3 is disclosed. Uses of CSF3 as a biomarker or therapeutic target for pulmonary fibrosis are disclosed. In addition, A pharmaceutical composition for treating pulmonary fibrosis containing an inhibitor of CSF3 and a treatment method of pulmonary fibrosis are disclosed.

### TECHNICAL FIELD

The present disclosure relates an anticancer adjuvant comprising an inhibitor of granulocyte colony-stimulating factor, also known as colony-stimulating factor 3 (CSF3), or a novel use of CSF3 as a biomarker and a therapeutic target for pulmonary fibrosis. In addition, the present disclosure relates to a pharmaceutical composition for treating pulmonary fibrosis, the composition comprising a CSF3 inhibitor.

### BACKGROUND ART

When tissue is damaged, a variety of cytokines are secreted, and a series of inflammatory reactions and healing processes occur. When the degree of damage is minor, the damaged area is subjected to a healing process while maintaining normal structure and function. However, when continuous stimulation or extreme damage occurs, the tissue loses its original function. As a result, with a variety of factors accumulated around the damaged area, fibrosis in which tissue becomes stiff occurs. In the process of fibrosis, extracellular matrix (ECM) components, such as collagen, fibronectin, and alpha-smooth muscle actin (α-SMA), are accumulated in the tissue, resulting in abnormal structure formation and functional impairment. As a result, the lung tissue becomes stiff, and smooth gas exchange fails to occur, resulting in symptoms, such as shortness of breath, and threats to the survival of all human beings.

Idiopathic pulmonary fibrosis is a disease in which chronic inflammatory cells infiltrate the alveolar walls and harden the lungs. The disease causes severe structural changes in lung tissue and gradually deteriorates lung functions, leading to death within an average of 3 years after diagnosis. Even though the cause of idiopathic pulmonary fibrosis has not yet been identified, the incidence is high in smokers and those over the age of 50. In addition, antidepressants, metal dust, wood dust, or inhalation of solvents have been reported as risk factors associated with the occurrence. In addition, since finding factors with a definite causal relationship in most patients is difficult, there is no effective treatment method yet. Recently, Rangarajan, S. and others have reported that metformin, an oral antidiabetic drug, is capable of alleviating the symptoms of pulmonary fibrosis in a bleomycin (BLM)-induced pulmonary fibrosis mouse model (Rangarajan, S., et al. Metformin reverses established lung fibrosis in a bleomycin model. Nat Med. 2018. 24, 1121-1127). However, in actual clinical trials, significant results were unobtainable. Therefore, discovering idiopathic pulmonary fibrosis-specific genes and developing therapeutic drugs targeting the genes are important.

### DISCLOSURE

### Technical Problem

The present disclosure has been made to solve the above problems. The inventors of the present disclosure have researched a therapeutic target capable of inhibiting idiopathic pulmonary fibrosis. As a result, based on the fact establishing that CSF3 can be a major target for the treatment of idiopathic pulmonary fibrosis, the present disclosure has been completed.

An objective of the present disclosure is to provide an anticancer adjuvant comprising a granulocyte-colony stimulating factor 3 (CSF3) inhibitor as an active ingredient.

In addition, another objective of the present disclosure is to provide a combination agent for anticancer comprising an anticancer agent and the anticancer adjuvant.

Furthermore, a further objective of the present disclosure is to provide a pharmaceutical composition for preventing or treating pulmonary fibrosis, the composition containing a granulocyte-colony stimulating factor 3 (CSF3) inhibitor as an active ingredient.

Moreover, still another objective of the present disclosure is to provide a method or use of the pharmaceutical composition for treating pulmonary fibrosis diseases.

However, the technical problem to be achieved by the present disclosure is not limited to the above-mentioned problems, and other problems not mentioned will be clearly understood by those skilled in the art from the description below.

### Technical Solution

In order to achieve the above objectives, the present disclosure provides a composition for combined anticancer therapy, comprising a Granulocyte-colony stimulating factor 3(CSF3) inhibitor as an active ingredient.

In one embodiment of the present disclosure, the composition may be capable of inhibiting a side effect of an anticancer drug.

In another embodiment of the present disclosure, the anticancer drug may be a bleomycin.

In still another embodiment of the present disclosure, the side effect of the anticancer drug may be an Idiopathic pulmonary fibrosis (IPF).

In still another embodiment of the present disclosure, the CSF3 inhibitor may be an CSF3 antibody or anti-CSF3 siRNA.

In still another embodiment of the present disclosure, the composition may be capable of inhibiting a differentiation of lung cells to myofibroblasts.

In still another embodiment of the present disclosure, the composition may be capable of inhibiting an Epithelial to Mesenchymal Transition (EMT).

In still another embodiment of the present disclosure, the composition may be capable of inhibiting an Extra Cellular Matrix remodeling (ECM remodeling).

In still another embodiment of the present disclosure, the inhibition of the differentiation into myofibroblasts may be induced by an inhibition of a α-Smooth Muscle Actin (α-SMA).

In still another embodiment of the present disclosure, the inhibition of the EMT may be induced by an inhibition of one or more protein selected from a group consisting of Fibronectin(FN), Vimentin(VIM), and ZEB1.

In still another embodiment of the present disclosure, the inhibition of the EMT may be induced by an inhibition of a STAT3 protein.

In still another embodiment of the present disclosure, the inhibition of ECM remodeling may be induced by an inhibition of one or more protein selected from a group consisting of Versican, Osteopontin (OPN), Collagen, and HAS3.

In still another embodiment of the present disclosure, the inhibition of ECM remodeling may be induced by an increase of a matrix metalloproteinase (MMP) protein.

In still another embodiment of the present disclosure, the inhibition of ECM remodeling may be induced by a decrease of a tissue inhibitors of metalloproteinase (TIMP) protein.

In still another embodiment of the present disclosure, the composition may be characterized in that it is administered simultaneously or sequentially with an anticancer drug.

In addition, the present disclosure provides a combination agent for anticancer, comprising an anticancer drug and the composition for combined anticancer therapy.

In addition, the present disclosure provides a pharmaceutical composition for preventing or treating a pulmonary fibrosis disease, comprising a Granulocyte-colony stimulating factor 3(CSF3) inhibitor as an active ingredient.

In one embodiment of the present disclosure, the pulmonary fibrosis disease may be induced by an anticancer drug.

In another embodiment of the present disclosure, the anticancer drug may be a bleomycin.

In still another embodiment of the present disclosure, the pulmonary fibrosis disease may comprise a myofibroblast hyperplasia of pulmonary cells or Idiopathic pulmonary fibrosis (IPF).

In still another embodiment of the present disclosure, the CSF3 inhibitor may be an anti-CSF3 antibody or anti-CSF3 siRNA.

In still another embodiment of the present disclosure, the composition may be capable of inhibiting a differentiation of lung cells into myofibroblasts.

In still another embodiment of the present disclosure, the composition may be capable of inhibiting an Epithelial to Mesenchymal Transition (EMT).

In still another embodiment of the present disclosure, the composition may be capable of inhibiting an Extra Cellular Matrix remodeling (ECM remodeling).

In still another embodiment of the present disclosure, the inhibition of the differentiation into myofibroblasts may be induced by an inhibition of a α-Smooth Muscle Actin (α-SMA).

In still another embodiment of the present disclosure, the inhibition of the EMT may be induced by an inhibition of one or more protein selected from a group consisting of Fibronectin (FN), Vimentin (VIM), and ZEB1.

In still another embodiment of the present disclosure, the inhibition of the EMT may be induced by an inhibition of a STAT3 protein.

In still another embodiment of the present disclosure, the inhibition of ECM remodeling is induced by an inhibition of one or more protein selected from a group consisting of Versican, Osteopontin (OPN), Collagen, and HAS3.

In still another embodiment of the present disclosure, the inhibition of ECM remodeling may be induced by an increase of a matrix metalloproteinase (MMP) protein.

In still another embodiment of the present disclosure, the inhibition of ECM remodeling may be induced by a decrease of a tissue inhibitors of metalloproteinase (TIMP) protein.

In addition, the present disclosure provides a method or an use of the pharmaceutical composition for treating pulmonary fibrosis disease.

### Advantageous Effects

When CSF3, whose expression was increased in idiopathic pulmonary fibrosis induced by bleomycin, an anticancer agent, is neutralized with a target antibody, markers of α-smooth muscle actin, collagen, and epithelial-mesenchymal transition (EMT) were confirmed to be decreased. Therefore, developing CSF3-targeting drugs is expected to treat pulmonary fibrosis by reducing epithelial-mesenchymal transition and accumulation of extracellular matrix components in lung epithelial cells of idiopathic pulmonary fibrosis patients.

However, the effects of the present disclosure are not limited to the above effects, and should be construed to include all effects that can be inferred from the detailed description of the present disclosure or the configuration of the disclosure described in the claims.

### DESCRIPTION OF DRAWINGS

FIG. 1A is a diagram illustrating the confirmation results of cytokine, chemokine, and a growth factor commonly expressed in patients with idiopathic pulmonary fibrosis (IPF) through the GEO database;

FIG. 1B is a diagram showing the results of selecting candidate factors having a high correlation with the disease in idiopathic pulmonary fibrosis patients by identifying the relevance of the factor selected in FIG. 1A;

FIG. 1C is a diagram showing the quantification results of the expression levels of the factors selected in FIG. 1B;

FIG. 1D is a diagram showing the imaging result of the expression levels of the factors selected in FIG. 1B;

FIG. 1E is a diagram showing changes in cytokine expression levels by performing tissue microarrays on idiopathic pulmonary fibrosis patients and healthy control group patients;

FIG. 1F is a diagram showing the CSF3 expression levels by performing tissue microarrays on idiopathic pulmonary fibrosis patients and healthy control group patients;

FIG. 1G is a diagram showing the confirmation results of CSF3 expression by performing immunohistochemical staining on lung tissues of idiopathic pulmonary fibrosis patients and healthy control group patients;

FIG. 1H is a diagram showing the confirmation results for the results confirmed in FIG. 1G with immunohistochemical staining score (IHC score);

FIG. 1I is a diagram showing the confirmation results of CSF3 expression by performing immunohistochemical staining on lung tissues of a mouse model in which idiopathic pulmonary fibrosis is induced by bleomycin (BLM) (hereinafter, referred to as BLM-induced idiopathic pulmonary fibrosis mouse model) and control group mice treated with PBS;

FIG. 1J is a diagram showing the confirmation results of CSF3 mRNA expression levels in lung tissues of a BLM-induced idiopathic pulmonary fibrosis mouse model and a normal control group;

FIG. 1K is a diagram showing the confirmation results of CSF3 levels by performing ELISA on lung tissues of a BLM-induced idiopathic pulmonary fibrosis mouse model and a normal control group;

FIG. 1L is a diagram showing the confirmation results that genes related to extracellular matrix (ECM) and epithelial-mesenchymal transition are highly expressed in idiopathic pulmonary fibrosis patients with high CSF3 expression, the results obtained by Gene Set Enrichment Analysis (GSEA);

FIG. 2A is a diagram showing the confirmation results of the expression of CSF family factors (CSF1, CSF2, and CSF3) by performing immunochemical staining on lung tissues of idiopathic pulmonary fibrosis patients and a BLM-induced idiopathic pulmonary fibrosis mouse model;

FIG. 2B is a diagram showing the confirmation results of the expression levels of CSF family factors in the lung tissues of idiopathic pulmonary fibrosis patients and a normal control group;

FIG. 2C is a diagram showing the confirmation results of the expression levels of CSF family factors by performing immunohistochemical staining on lung tissues of a BLM-induced idiopathic pulmonary fibrosis mouse model and a normal control group;

FIG. 2D is a diagram showing the confirmation results of the expression levels of CSF family factors by performing Western blotting on lung tissues of a BLM-induced idiopathic pulmonary fibrosis mouse model and a normal control group;

FIG. 2E is a diagram showing the confirmation results of the mRNA expression levels of CSF family factors in lung tissues of a BLM-induced idiopathic pulmonary fibrosis mouse model and a normal control group;

FIG. 3A is a diagram illustrating an experimental design result for confirming the transdifferentiation of lung epithelial cells into myofibroblasts in a BLM-induced idiopathic pulmonary fibrosis mouse model;

FIG. 3B is a diagram showing the confirmation results of transdifferentiation of lung epithelial cells into myofibroblasts by epithelial-mesenchymal transition (EMT) by performing immunohistochemical staining (H&E staining, Col1a1 staining, α-SMA staining, Serius red staining, and polarizing microscope observation) on lung tissues of a BLM-induced idiopathic pulmonary fibrosis mouse model and a normal control group;

FIG. 3C is a diagram showing the confirmation results of hydroxyproline contents in Beas-2b cells and bleomycin-treated Beas-2b cells through a hydroxyproline assay;

FIG. 3D is a diagram showing the confirmation results of the expression levels of α-SMA, Col1a1, osteopontin (OPN), versican (VER), vimentin (VIM), fibronectin (FN), and Snail comparing with β-actin. The expression level is confirmed by performing Western blotting on Beas-2b cells and bleomycin-treated Beas-2b cells;

FIG. 3E is a diagram showing the confirmation result of the mRNA expression levels of α-SMA and COL1A1 by performing RT-qPCR on Beas-2b cells and bleomycin-treated Beas-2b cells;

FIG. 3F is a diagram showing results of the expression levels of factors related to epithelial-mesenchymal transition (EMT) in idiopathic pulmonary fibrosis patients by GSEA;

FIG. 3G is a diagram showing the observation results after performing idiopathic pulmonary fibrosis marker (α-SMA and Col1a1) staining and DAPI staining on Beas-2b in which idiopathic pulmonary fibrosis is induced by bleomycin and Beas-2b of a normal control group;

FIG. 3H is a diagram showing the confirmation results of performing F-actin staining and DAPI staining on Beas-2b in which idiopathic pulmonary fibrosis is induced by bleomycin and Beas-2b of a normal control group to confirm whether cells change into fusiform cells;

FIG. 3I is a diagram showing the confirmation result of the cell ratio in which migration or invasion occurred in lung tissues of a BLM-induced idiopathic pulmonary fibrosis mouse model and a normal control group;

FIG. 3J is a diagram showing the confirmation results of the expression of markers (N-cad, E-cad, VIM, and FN) related to epithelial-mesenchymal transition (EMT) by performing immunohistochemical staining on lung tissues of a BLM-induced idiopathic pulmonary fibrosis mouse model and a normal control group;

FIG. 3K is a diagram showing the confirmation results of the mRNA expression levels of N-cad and FN by performing RT-qPCR on lung tissues of a BLM-induced idiopathic pulmonary fibrosis mouse model and a normal control group;

FIG. 4A is a diagram showing the results of the mRNA expression levels of α-SMA, Col1a1, OPN, VER, Has3, N-cad, FN, VIM, Zeb1, and Slug in Beas-2b cells, BLM-treated Beas-2b cells, and an experimental group in which the BLM-treated Beas-2b cells are treated with si-CSF3;

FIG. 4B is a diagram showing the confirmation results of the expression levels of α-SMA, Col1a1, OPN, VER, Has3, N-cad, FN, VIM, Zeb1, and Slug by performing Western blotting on Beas-2b cells, BLM-treated Beas-2b cells, and an experimental group in which the BLM-treated Beas-2b cells are treated with si-CSF3;

FIG. 4C is a diagram showing the confirmation results of the mRNA expression levels of CSF3, α-SMA, Col1a1, OPN, VER, Has3, N-cad, VIM, Snail, and Slug in Beas-2b cells and Beas-2b cells in which CSF3 is overexpressed;

FIG. 4D is a diagram showing the confirmation results of the expression of α-SMA, Col1a1, OPN, Has3, FN, N-cad, VIM, Slug, MYC, CSF3, and β-actin by performing Western blotting on Beas-2b cells and Beas-2b cells in which CSF3 is overexpressed;

FIG. 4E is a diagram showing the confirmation results of the mRNA expression of α-SMA, Col1a1, OPN, VER, FN, N-cad, and Slug in Beas-2b cells and Beas-2b cells treated with recombinant human CSF3 (rhCSF3);

FIG. 4F is a diagram showing the confirmation results of the expression of α-SMA, Col1a1, OPN, VER, Has3, VIM, Snail, Slug, and β-actin by performing Western blotting on Beas-2b cells and Beas-2b cells treated with recombinant human CSF3 (rhCSF3);

FIG. 5A is a diagram showing the screening results of epithelial-mesenchymal transition (EMT) downstream signaling pathway factors by treating a lung epithelial cell line Beas-2b with rhCSF3;

FIG. 5B is a diagram showing the confirmation results that the activity of STAT3 induced by BLM is decreased as the expression of CSF3 receptor (CSF3R) is inhibited, the results confirmed by performing Western blotting;

FIG. 5C is a diagram showing the co-immunoprecipitation (co-IP) results indicating that CSF3 receptor (CSF3R) and STAT3 directly bind to each other;

FIG. 5D is a diagram showing the in situ PLA assay results indicating that CSF3 receptor (CSF3R) and STAT3 directly bind to each other;

FIG. 5E is a diagram showing the in situ PLA assay results indicating that CSF3 receptor (CSF3R) and STAT3 directly bind to each other;

FIG. 5F is a diagram showing the confirmation results of the p-Stat3 expression in lung tissues of a BLM-induced idiopathic pulmonary fibrosis mouse model and a control group;

FIG. 5G is a diagram showing the confirmation results of the mRNA expression levels of CSF3, α-SMA, Col1a1, OPN, Has3, N-cad, VIM, Zeb1, and Snail in a control group (treated with si-cont), an experimental group treated with si-cont and rhCSF3, and an experimental group treated with si-STAT3 and rhCSF3;

FIG. 5H is a diagram showing the confirmation results of the expression levels of CSF3, α-SMA, Col1a1, OPN, Has3, N-cad, VIM, Zeb1, and Snail by performing Western blotting on a control group (treated with si-cont), an experimental group treated with si-cont and rhCSF3, and an experimental group treated with si-STAT3 and rhCSF3;

FIG. 5I is a diagram showing the confirmation results of the mRNA expression levels of CSF3R, CSF3, α-SMA, Col1a1, OPN, VER, Has3, N-cad, FN, VIM, Zeb1, Snail, and Slug in a control group (treated with DMSO), an experimental group treated with rhCSF3, and an experimental group treated with rhCSF3 and a STAT3 inhibitor (Stat3i);

FIG. 5J is a diagram showing the confirmation results of the expression levels of CSF3R, CSF3, α-SMA, Col1a1, OPN, VER, Has3, N-cad, FN, VIM, Zeb1, Snail, and Slug by performing Western blotting on a control group (treated with DMSO), an experimental group treated with rhCSF3, and an experimental group treated with rhCSF3 and a STAT3 inhibitor (Stat3i);

FIG. 5K is a diagram showing the confirmation results of the mRNA expression levels of CSF3, α-SMA, Col1a1, OPN, VER, Has3, N-cad, VIM, Snail, and Slug in a control group, a CSF3-overexpressed group (CSF3 OE), and an experimental group in which the CSF3-overexpressed group is treated with si-STAT3;

FIG. 5L is a diagram showing the confirmation results of the expression levels of CSF3, α-SMA, Col1a1, OPN, VER, Has3, N-cad, VIM, Snail, and Slug by performing Western blotting on a control group, a CSF3-overexpressed group (CSF3 OE), and an experimental group in which the CSF3-overexpressed group is treated with si-STAT3;

FIG. 6A is a diagram illustrating an experimental design for confirming the prevention of pulmonary fibrosis with a CSF3-neutralizing antibody in a BLM-induced idiopathic pulmonary fibrosis mouse model;

FIG. 6B is a diagram showing the confirmation results of the occurrence of pulmonary fibrosis by performing H&E staining on a control group, a BLM-induced idiopathic pulmonary fibrosis mouse model, and a mouse model treated with a CSF3-neutralizing antibody;

FIG. 6C is a diagram showing the confirmation results of the occurrence of pulmonary fibrosis by performing Col1a1 staining and α-SMA staining on a control group, a BLM-induced idiopathic pulmonary fibrosis mouse model, and a mouse model treated with a CSF3-neutralizing antibody;

FIG. 6D is a diagram showing the confirmation results of the occurrence of pulmonary fibrosis by performing Sirius red staining on a control group, a BLM-induced idiopathic pulmonary fibrosis mouse model, and a mouse model treated with a CSF3-neutralizing antibody;

FIG. 6E is a diagram showing the confirmation results of the mRNA expression levels of FN, VIM, E-cad, and N-cad by performing RT-qPCR on lung tissues of a control group, a BLM-induced idiopathic pulmonary fibrosis mouse model, and a mouse model treated with a CSF3-neutralizing antibody;

FIG. 6F is a diagram showing the confirmation results of the expression levels of FN, VIM, E-cad, and N-cad by performing Western blotting on lung tissues of a control group, a BLM-induced idiopathic pulmonary fibrosis mouse model, and a mouse model treated with a CSF3-neutralizing antibody;

FIG. 6G is a diagram showing the confirmation results of the expression levels of FN, VIM, E-cad, and N-cad by performing immunohistochemical staining on lung tissues of a control group, a BLM-induced idiopathic pulmonary fibrosis mouse model, and a mouse model treated with a CSF3-neutralizing antibody;

FIG. 6H is a diagram showing the confirmation results of the probability of survival over time of a control group (5 mice), a BLM-induced idiopathic pulmonary fibrosis mouse model (7 mice), and a CSF3 knock-out mouse model (5 mice);

FIG. 7A is a diagram illustrating an experimental design for confirming the therapeutic effect of a CSF3-neutralizing antibody on pulmonary fibrosis in a BLM-induced idiopathic pulmonary fibrosis mouse model;

FIG. 7B is a diagram showing the confirmation results of the effect of improving pulmonary fibrosis by performing H&E staining on lung tissues of a control group, a BLM-induced idiopathic pulmonary fibrosis mouse model, and a mouse model treated with a CSF3-neutralizing antibody;

FIG. 7C is a diagram showing the confirmation results of the effect of improving pulmonary fibrosis by performing Sirius red staining on a control group, a BLM-induced idiopathic pulmonary fibrosis mouse model, and a mouse model treated with CSF3-neutralizing antibodies;

FIG. 7D is a diagram showing the confirmation results of the occurrence of pulmonary fibrosis by performing Col1a1 staining and α-SMA staining on a control group, a BLM-induced idiopathic pulmonary fibrosis mouse model, and a mouse model treated with a CSF3-neutralizing antibody;

FIG. 7E is a diagram showing the confirmation results of the expression of p-Stat3, FN, VIM, E-cad, and N-cad by performing immunohistochemical staining on lung tissues of a control group, a BLM-induced idiopathic pulmonary fibrosis mouse model, and a mouse model treated with a CSF3-neutralizing antibody;

FIG. 7F is a diagram showing the confirmation results of the expression of α-SMA, Col1a1, OPN, Has3, FN, Snail, CSF3, Stat3, β-actin, p-Stat3, FN, VIM, E-cad, and N-cad by performing Western blotting on lung tissues of a control group, a BLM-induced idiopathic pulmonary fibrosis mouse model, and a mouse model treated with a CSF3-neutralizing antibody;

FIG. 7G is a diagram showing the confirmation results of hydroxyproline contents in a control group, a BLM-induced idiopathic pulmonary fibrosis mouse model, and a mouse model treated with a CSF3-neutralizing antibody, the results obtained through a hydroxyproline assay;

FIG. 7H is a diagram showing the confirmation results of CSF3 expression by performing ELISA on a control group, a BLM-induced idiopathic pulmonary fibrosis mouse model, and a mouse model treated with a CSF3-neutralizing antibody;

FIG. 7I is a diagram showing the confirmation results of the expression levels of TGF-β, ρ-AMPK, β-actin by performing Western blotting on a control group, a BLM-induced idiopathic pulmonary fibrosis mouse model, and a mouse model treated with a CSF3-neutralizing antibody;

FIG. 7J is a diagram showing the confirmation results of relative TGF-β expression levels in a control group, a BLM-induced idiopathic pulmonary fibrosis mouse model, and a mouse model treated with a CSF3-neutralizing antibody;

FIG. 7K is a diagram showing the confirmation results of expression by performing α-SMA staining, Col1a1 staining, F-actin staining, and DAPI staining on lung tissues of a control group, a BLM-induced idiopathic pulmonary fibrosis mouse model, and a mouse model treated with a CSF3-neutralizing antibody;

FIG. 7L is a diagram showing the confirmation results of the probability of survival over time of a control group (5 mice), a BLM-induced idiopathic pulmonary fibrosis mouse model (6 mice), and a CSF3 knock-out mouse model (6 mice);

FIG. 8A is a diagram showing the confirmation results of the mRNA expression levels of α-SMA and COL1A1 in a control group (si-cont-treated group), an experimental group in which a BLM-induced idiopathic pulmonary fibrosis mouse model is treated with si-cont, and an experimental group in which a mouse model is treated with CSF family inhibitors (si-CSF1, si-CSF2, and si-CSF3);

FIG. 8B is a diagram showing the confirmation results of the mRNA expression levels of OPN, VER, and Has3 in a control group (si-cont treated group), an experimental group in which a BLM-induced idiopathic pulmonary fibrosis mouse model is treated with si-cont, and an experimental group in which a mouse model is treated with CSF family inhibitors (si-CSF1, si-CSF2, and si-CSF3);

FIG. 8C is a diagram showing the confirmation results of the expression levels of α-SMA, Col1a1, OPN, VER, Has3, N-cad, Zeb1, Snail, Slug, and β-actin by performing Western blotting on a control group (si-cont-treated group), an experimental group in which a BLM-induced idiopathic pulmonary fibrosis mouse model is treated with si-cont, and an experimental group in which a mouse model is treated with CSF family inhibitors (si-CSF1, si-CSF2, and si-CSF3)

FIG. 8D is a diagram illustrating an experimental design for confirming the effect of treating pulmonary fibrosis with CSF3 family neutralizing antibodies in a BLM-induced idiopathic pulmonary fibrosis mouse model;

FIG. 8E is a diagram showing the confirmation results of the expression levels of Col1a1 and α-SMA by performing immunohistochemical staining on a control group, a BLM-induced idiopathic pulmonary fibrosis mouse model, and an experimental group in which a mouse model is treated with CSF family antibodies (si-CSF1, si-CSF2, and si-CSF3);

FIG. 8F is a diagram showing the confirmation results of the expression levels of N-cad, E-cad, VIM, and FN by performing immunohistochemical staining on a control group, a BLM-induced idiopathic pulmonary fibrosis mouse model, and an experimental group in which a mouse model is treated with CSF family antibodies (si-CSF1, si-CSF2, and si-CSF3);

FIG. 8G is a diagram showing the confirmation results of the expression levels of α-SMA, Col1a1, OPN, VER, FN, and β-actin by performing Western blotting on a control group, a BLM-induced idiopathic pulmonary fibrosis mouse model, and an experimental group in which a mouse model is treated with CSF family antibodies (si-CSF1, si-CSF2, and si-CSF3);

FIG. 8H is a diagram showing the confirmation results of the mRNA expression levels of α-SMA, N-cad, Col1a1, and FN in a control group, a BLM-induced idiopathic pulmonary fibrosis mouse model, and an experimental group in which a mouse model is treated with CSF family antibodies (si-CSF1, si-CSF2, and si-CSF3);

FIG. 8I is a diagram showing the confirmation results of changes in body weight over time in a control group, a BLM-induced idiopathic pulmonary fibrosis mouse model, and an experimental group in which a mouse model is treated with CSF family antibodies (si-CSF1, si-CSF2, si-CSF3);

FIG. 9A is a diagram showing the confirmation results of the expression levels of MMP2, MMP9, and MMP13 by performing immunohistochemical staining on a control group, a BLM-induced idiopathic pulmonary fibrosis mouse model, and an experimental group in which a mouse model is treated with a CSF3 antibody;

FIG. 9B is a diagram showing the confirmation results of the expression levels of MMP2, MMP13, and β-actin by performing Western blotting on a control group, a BLM-induced idiopathic pulmonary fibrosis mouse model, and an experimental group in which a mouse model is treated with a CSF3 antibody;

FIG. 9C is a diagram showing the confirmation results of the mRNA expression levels of MMP2, MMP9, and MMP13 in a control group, a BLM-induced idiopathic pulmonary fibrosis mouse model, and an experimental group in which a mouse model is treated with a CSF3 antibody;

FIG. 9D is a diagram showing the confirmation results of the expression of TIMP-1 and TIMP-2 by performing immunohistochemical staining on a control group, a BLM-induced idiopathic pulmonary fibrosis mouse model, and an experimental group in which a mouse model is treated with a CSF3 antibody;

FIG. 9E is a diagram showing the confirmation results of the expression levels of TIMP-1, TIMP-2, and β-actin by performing Western blotting on a control group, a BLM-induced idiopathic pulmonary fibrosis mouse model, and an experimental group in which a mouse model is treated with a CSF3 antibody;

FIG. 9F is a diagram showing the confirmation results of mRNA expression level of TIMP-1 in a control group, a BLM-induced idiopathic pulmonary fibrosis mouse model, and an experimental group in which a mouse model is treated with a CSF3 antibody;

FIG. 10A is a diagram showing an experimental design for comparing the effects of metformin, a TGF-β antibody, or a CSF3 antibody for treating idiopathic pulmonary fibrosis in a BLM-induced idiopathic pulmonary fibrosis mouse model.

FIB. 10B is a diagram showing the confirmation results of the effects of treating idiopathic pulmonary fibrosis after performing H&E staining, Sirius red staining, and trichrome staining on a control group, a BLM-induced idiopathic pulmonary fibrosis mouse model, and an experimental group in which a mouse model is treated with metformin, a TGF-β antibody, or a CSF3 antibody;

FIG. 10C is a diagram showing the confirmation results of confirming the expression of α-SMA, COL1A1, FN, and CSF3 by performing immunohistochemical staining on a control group, a BLM-induced idiopathic pulmonary fibrosis mouse model, and an experimental group in which a mouse model is treated with metformin, a TGF-β antibody, or a CSF3 antibody;

FIG. 10D is a diagram showing the confirmation results of the mRNA expression levels of α-SMA and COL1A1 in a control group, a BLM-induced idiopathic pulmonary fibrosis mouse model, and an experimental group in which a mouse model is treated with a CSF3 antibody;

FIG. 10E is a diagram showing the confirmation results of the mRNA expression levels of α-SMA, COL1A1, OPN, HAS3, and β-actin by performing Western blotting on a control group, a BLM-induced idiopathic pulmonary fibrosis mouse model, and an experimental group in which a mouse model is treated with metformin, a TGF-β antibody, or a CSF3 antibody;

FIG. 10F is a diagram showing the observation results of the progress for eight days in a control group, a BLM-induced idiopathic pulmonary fibrosis mouse model, and an experimental group in which a mouse model is treated with metformin, a TGF-β antibody, or a CSF3 antibody;

FIG. 10G is a diagram showing the confirmation results of changes in body weight for twenty-one days by observing a control group, a BLM-induced idiopathic pulmonary fibrosis mouse model, and an experimental group in which a mouse model is treated with metformin, a TGF-β antibody, or a CSF3 antibody;

FIG. 10H is a diagram showing the confirmation results of the mRNA expression levels of α-SMA, Col1a1, OPN, VER, Has3, N-cad, FN, VIM, and Zeb1 in a control group, a BLM-treated lung epithelial cell line Beas-2b, and an experimental group in which the BLM-treated lung epithelial cell line Beas-2b is treated with metformin or a CSF3 antibody;

FIG. 10I is a diagram showing the confirmation results of the expression levels of α-SMA, Col1a1, OPN, VER, Has3, N-cad, FN, VIM, Zeb1, Snail, p-AMPK, and β-actin by performing Western blotting on a control group, a BLM-treated lung epithelial cell line Beas-2b, and an experimental group in which the BLM-treated lung epithelial cell line Beas-2b is treated with metformin or a CSF3 antibody;

FIG. 10J is a diagram showing the confirmation results of the mRNA expression levels of α-SMA, Col1a1, OPN, VER, Has3, N-cad, FN, VIM, and Zeb1 in a control group, a BLM-treated lung epithelial cell line Beas-2b, and an experimental group in which the BLM-treated lung epithelial cell line Beas-2b is treated with a TGF-β antibody or a CSF3 antibody;

FIG. 10K is a diagram showing the confirmation results of the expression levels of α-SMA, Col1a1, OPN, VER, Has3, N-cad, FN, VIM, Zeb1, Snail, p-AMPK, and β-actin by performing Western blotting on a control group, a BLM-treated lung epithelial cell line Beas-2b, and an experimental group in which the BLM-treated lung epithelial cell line Beas-2b is treated with a TGF-β antibody or a CSF3 antibody;

FIG. 10L is a diagram showing the confirmation results of the mRNA expression levels of α-SMA, Col1a1, OPN, VER, Has3, N-cad, FN, and VIM in a control group, a BLM-treated lung epithelial cell line Beas-2b, and an experimental group in which the BLM-treated lung epithelial cell line Beas-2b is treated with si-TGF-β or si-CSF3;

FIG. 10M is a diagram showing the confirmation results of the expression levels of α-SMA, Col1a1, OPN, VER, Has3, N-cad, FN, Snail, Slug, and β-actin by performing Western blotting on a control group, a BLM-treated lung epithelial cell line Beas-2b, and an experimental group in which BLM-treated lung epithelial cell line Beas-2b is treated with si-TGF-β or si-CSF3;

FIG. 10N is a diagram showing the filming results of a control group not being treated with an antibody and a BLM-induced idiopathic pulmonary fibrosis mouse model over time (day 1, day 4, day 7, day 15, and day 20);

FIG. 10O is a diagram showing the filming results of mouse model conditions when three days and eight days have elapsed after treating a BLM-induced idiopathic pulmonary fibrosis mouse model with metformin, a TGF-β antibody, or a CSF3 antibody;

FIG. 11A is a diagram showing an experimental design for comparing the therapeutic effect of pirfenidone or a CSF3 antibody on idiopathic pulmonary fibrosis in a BLM-induced idiopathic pulmonary fibrosis mouse model;

FIG. 11B is a diagram showing the observation results of idiopathic pulmonary fibrosis by performing H&E staining and Sirius red staining on a control group, a BLM-induced idiopathic pulmonary fibrosis mouse model, and an experimental group in which a mouse model is treated with pirfenidone or a CSF3 antibody;

FIG. 11C is a diagram showing the confirmation results of the expression of α-SMA, COL1, and CSF3 by performing immunohistochemical staining on a control group, a BLM-induced idiopathic pulmonary fibrosis mouse model, and an experimental group in which a mouse model is treated with pirfenidone or a CSF3 antibody;

FIG. 11D is a diagram showing the confirmation results of the expression levels of α-SMA, COL1, and CSF3 in a control group, a BLM-induced idiopathic pulmonary fibrosis mouse model, and an experimental group in which a mouse model is treated with pirfenidone or a CSF3 antibody;

FIG. 11E is a diagram showing the observation results of eight days over time for a control group, a BLM-induced idiopathic pulmonary fibrosis mouse model, and an experimental group in which a mouse model is treated with pirfenidone or a CSF3 antibody;

FIG. 11F is a diagram showing the confirmation results of the expression levels of α-SMA, Col1a1, and β-actin by performing Western blotting on a control group, a BLM-induced idiopathic pulmonary fibrosis mouse model, and an experimental group in which a mouse model is treated with pirfenidone or a CSF3 antibody; and

FIG. 11G is a diagram showing the confirmation results of the expression levels of α-SMA, Col1a1, and β-actin in a control group, a BLM-induced idiopathic pulmonary fibrosis mouse model, and an experimental group in which a mouse model is treated with pirfenidone or a CSF3 antibody.

### BEST MODE

The inventors of the present disclosure confirmed that when CSF3, whose expression was increased in idiopathic pulmonary fibrosis induced by bleomycin, an anticancer agent, is neutralized with a target antibody, markers of α-smooth muscle actin, collagen, and epithelial-mesenchymal transition (EMT) were confirmed to be decreased. As a result, the present disclosure has been completed.

Hence, the present disclosure provides an anticancer adjuvant comprising a granulocyte-colony stimulating factor 3 (CSF3) inhibitor as an active ingredient.

The anticancer adjuvant may be a component that inhibits idiopathic pulmonary fibrosis among the side effects of anticancer agents. The anticancer agent may be bleomycin, but is not limited thereto. The anticancer adjuvant may inhibit the differentiation of normal lung cells into myofibroblasts when pulmonary fibrosis develops, which may be attributable to inhibition of α-Smooth Muscle Actin (α-SMA).

As used herein, “pulmonary fibrosis” is a respiratory disease in which lung tissue hardens and leads to severe respiratory disorders. Hardening of the lungs means excessive accumulation of fibrous connective tissue, and this process is called fibrosis. As fibrosis develops, the lung wall thickens, resulting in a decrease in an amount of oxygen supplied to the blood. As a result, the patients may constantly feel short of breath. In addition, it is reported that there is no method to repair lung tissue with advanced fibrosis. In one embodiment, pulmonary fibrosis may be idiopathic pulmonary fibrosis, pulmonary inflammatory fibrotic disease, chronic obstructive pulmonary disease, or fibrotic disease caused by asthma. Specifically, pulmonary fibrosis may be idiopathic pulmonary fibrosis.

In another aspect, the present disclosure relates to the anticancer adjuvant for treating pulmonary fibrosis, the adjuvant containing a substance that inhibits the expression or activity of CSF3. In one embodiment, the anti-cancer adjuvant of the present disclosure may inhibit the expression of α-Smooth Muscle Actin (α-SMA) in lung cells. In another embodiment, the anticancer adjuvant of the present disclosure may inhibit collagen expression in lung tissue. In a further embodiment, the anticancer adjuvant of the present disclosure may inhibit the epithelial-mesenchymal transition (EMT) of lung epithelial cells. Specifically, the inhibition of epithelial-mesenchymal transition may be induced by inhibiting the expression of one or more types of protein selected from the group consisting of fibronectin (FN), vimentin (VIM), N-cad, and ZEB1, or may be induced by the inhibition of STATS protein.

As used herein, “epithelial-mesenchymal transition” refers to a process in which epithelial cells lose their cell polarity and cell-to-cell adhesion while gaining migration and invasiveness properties to become mesenchymal stem cells, which are pluripotential stroma cells capable of differentiating into various cell types. The epithelial-mesenchymal transition is essential for numerous developmental processes, comprising mesoderm formation and neural tube formation, and is observed during wound healing, organ fibrosis, and cancer metastasis. Therefore, pulmonary fibrosis can be treated by inhibiting the epithelial-mesenchymal transition of the lung epithelial cells.

As described above, the anticancer adjuvant of the present disclosure inhibits the expression of α-Smooth Muscle Actin (α-SMA) in the lung cells, inhibits the expression of collagen in the lung tissue, and/or inhibits epithelial-mesenchymal transition in the lung epithelial cells. As a result, the accumulation of extracellular matrix components may be ultimately reduced, thereby effectively treating pulmonary fibrosis.

The anticancer adjuvant of the present disclosure may be administered simultaneously or sequentially with an anticancer drug, but is not limited thereto.

In the anticancer adjuvant according to the present disclosure, the CSF3 inhibitor may be a substance capable of inhibiting expression or activity of CSF3. Specific examples of the CSF3 inhibitor may comprise anti-CSF3 siRNA, an anti-CSF3 antibody, anti-CSF3 shRNA, anti-CSF3 antisense nucleic acid, guide RNA (gRNA) and CRISPR/Cas9, an anti-CSF3 small molecule compound, or an anti-CSF3 antibody. Preferably, the CSF3 inhibitor is the anti-CSF3 siRNA or anti-CSF3 antibody, but is not limited thereto.

Specifically, the substance that inhibits the CSF3 expression may be the anti-CSF3 siRNA, anti-CSF3 shRNA, anti-CSF3 antisense nucleic acid, guide RNA (gRNA), CRISPR/Cas9, and the like that can specifically bind to CSF3 mRNA. In addition, the substance that inhibits the CSF3 activity may be the anti-CSF3 small molecule compound, anti-CSF3 antibody, or the like that specifically binds to CSF3 to inhibit the CSF3 activity.

Short hairpin RNA or small hairpin RNA (shRNA) is used to overcome the disadvantages of the high cost of siRNA biosynthesis and short-term maintenance of RNA interference effect due to low cell transfection efficiency. The shRNA may be introduced into cells and expressed by using adenovirus, lentivirus, and plasmid expression vector systems from RNA polymerase promoter. Such shRNA is widely known to be converted into siRNAs with precise structures by siRNA processing enzymes (Dicer or Rnase 2) present in cells to induce target gene silencing.

Antisense nucleic acid refers to DNA, RNA, or derivatives thereof containing a nucleic acid sequence complementary to a specific mRNA sequence. The antisense nucleic acid binds to the complementary sequence in mRNA for inhibition of translation of mRNA into protein, translocation into cytoplasm, maturation, or essential activity to any other vital activities for overall biological functions.

The antibody capable of specifically binding to CSF3 comprises a monoclonal antibody, a chimeric antibody corresponding thereto, a humanized antibody, and a human antibody, and may also comprise antibodies already known in the art, in addition to novel antibodies. As long as the antibody specifically binds to CSF3, the antibody comprises functional fragments of an antibody molecule as well as full-length forms of two heavy chains and two light chains. The functional fragment of an antibody molecule means a fragment having at least an antigen-binding function, and comprises Fab, F(ab′), F(ab′)2, Fv, and the like.

The inventors of the present disclosure have confirmed through specific examples that the anticancer adjuvant of the present disclosure, which contains the CSF3 inhibitor, is capable of preventing or treating idiopathic pulmonary fibrosis induced by anticancer agents, comprising bleomycin.

In one embodiment of the present disclosure, by using a human tissue microarray, a GEO database, and a BLM-induced pulmonary fibrosis mouse model, the expression of CSF3 was observed to be higher in pulmonary fibrosis tissue than in normal lung tissues. In addition, it was confirmed that, among CSF family (CSF1, CSF2, and CSF3), only CSF3 expression was high (see Example 2).

In another embodiment of the present disclosure, it was confirmed that pulmonary fibrosis was induced by the EMT and the differentiation of myofibroblasts occurring in the pulmonary fibrosis mouse model having high expression level of CSF3 and in the lung epithelial cell line Beas-2b having high expression level of CSF3 as described above. When being treated with the CSF3 inhibitor, it was confirmed that pulmonary fibrosis and EMT were inhibited (see Example 3). It was confirmed that these are carried out by a pathway based on fibronectin (FN), vimentin (VIM), ZEB1, and STAT3 protein (see Example 4).

In a further embodiment of the present disclosure, when the bleomycin-induced pulmonary fibrosis mouse model is pretreated with the CSF3 inhibitor, it was confirmed that pulmonary fibrosis was able to be prevented (see Example 5). Even when pulmonary fibrosis was advanced, it was confirmed that pulmonary fibrosis was able to be treated when treated with the CSF3 inhibitor, and this effect was significantly obtained only in the case of inhibiting CSF3 among the CSF family (see Example 6).

In a further embodiment of the present disclosure, it was confirmed that the inhibitor targeting CSF3 of the present disclosure had a significantly higher therapeutic effect on pulmonary fibrosis than a TGF-β antibody or metformin, previously known as a therapeutic agent. In addition, it was confirmed through specific experiments that the CSF3 inhibitor had a significantly higher therapeutic effect on pulmonary fibrosis than pirfenidone, a clinically used pulmonary fibrosis drug approved by the FDA (see Example 8).

Through the above results, the inventors of the present disclosure have confirmed that CSF3 of the present disclosure can be a biomarker for the diagnosis of pulmonary fibrosis and a new target for the treatment of pulmonary fibrosis. And it was also confirmed that pulmonary fibrosis can be treated by inhibiting the expression or activity of CSF3.

In another aspect of the present disclosure, the present disclosure provides a combination agent for anticancer comprising an anticancer drug and the anticancer adjuvant.

In a further aspect of the present disclosure, the present disclosure provides a pharmaceutical composition for preventing or treating pulmonary fibrosis, the composition containing a granulocyte-colony stimulating factor 3 (CSF3) inhibitor as an active ingredient.

A method of injecting the substance that inhibits the expression or activity of CSF3 into pulmonary fibrosis cells may be implemented in various forms, such as a method using a liposome or vector system, which is a genetic engineering technology. Alternatively, a conventional manner in administrating the pharmaceutical formulation in vivo may be used after preparing a pharmaceutical formulation.

The pharmaceutical composition, according to the present disclosure, may further comprise a pharmaceutically acceptable carrier. Examples of the pharmaceutically acceptable carrier, typically used during formulation, comprises a saline solution, sterile water, a Ringer's solution, buffered saline, cyclodextrin, a dextrose solution, a maltodextrin solution, glycerol, ethanol, liposome, and the like, but not limited thereto. The carrier may further comprise other commonly known additives, such as an antioxidant, a buffer solution, and the like, as needed. In addition, a diluent, a dispersant, a surfactant, a binder, a lubricant, and the like may be additionally added to be formulated into an injectable formulation, such as an aqueous solution, a suspension, an emulsion, etc., a pill, a capsule, a granule, or a tablet. Regarding the pharmaceutically acceptable carrier and formulation that are appropriate, each component is preferably formulated using methods disclosed in Remington's Pharmaceutical Sciences (19th edition, 1995). In the present disclosure, the formulation of the pharmaceutical composition is not particularly limited, but the pharmaceutical composition may be formulated in the form of an injection, an inhalant, a dermatologic agent, an oral ingestible agent, or the like.

The pharmaceutical composition of the present disclosure may be administered orally or parenterally (for example, intravenous administration, subcutaneous administration, intranasal administration, percutaneous administration, and airway administration) depending on the desired method. A dosage of the pharmaceutical composition may vary according to the condition and body weight of a patient, the severity of the disease, a drug type, an administration route, and an administration time, and may be appropriately selected by those skilled in the art.

The composition, according to the present disclosure, is administered in a therapeutically effective dose. As used herein, the “pharmaceutically effective dose” means an amount sufficient to treat a disease at a reasonable benefit-risk ratio applicable to medical treatment. The effective dose level may be determined according to types of disease, severity, the activity of a drug, sensitivity to a drug, an administration time, an administration route and rate of release, duration of treatment, factors comprising concurrent medications, and other factors well-known in the medical field. The pharmaceutical composition, according to the present disclosure, may be administered as a single therapeutic agent or administered in combination with other therapeutic agents, and may be administered sequentially or simultaneously with conventional therapeutic agents, once or multiple times. Considering all of the above factors, administering an amount capable of obtaining the maximum effect with the minimum amount without side effects is critical, which can be easily determined by those skilled in the art.

Specifically, the effective dose of the composition, according to the present disclosure, may vary depending on the age, gender, and weight of a patient. Typically, the effective dose of the composition may be in a range of 0.001 mg to 150 mg per 1 kg of body weight, and preferably in the range of 0.01 mg to 100 mg daily or every other day once or three times per day. However, the dose may increase or decrease depending on an administration route, the severity of pulmonary fibrosis, gender, weight, age, and the like, so the dosage does not limit the scope of the present disclosure in any way.

On the other hand, in order to identify the mechanism by which pulmonary fibrosis induced by bleomycin (BLM) treatment is restored to normal lung tissue by a CSF3-neutralizing antibody, the inventors of the present disclosure analyzed the expression of matrix metalloproteinase (MMP) and tissue inhibitors of metalloproteinase (TIMP) related to ECM degradation. As a result, as confirmed in the following embodiments, it was identified that a CSF3-neutralizing antibody exhibited a therapeutic effect on pulmonary fibrosis by regulating the expression of MMPs (MMP2, MMP9, MMP13, and the like) and TIMPs (TIMP-1, TIMP-2, and the like) in the BLM-induced pulmonary fibrosis mouse model. In addition, the inventors of the present disclosure have further identified that STATS is a major factor in downstream signaling mechanism that induces epithelial-mesenchymal transition (EMT) induced by CSF3 in a lung epithelial cell line.

In a further aspect, the present disclosure provides a method for preventing or treating pulmonary fibrosis, the method comprising administering the composition to a subject.

As used herein, the term “prevention” refers to all activities that inhibit pulmonary fibrosis or delay the onset of pulmonary fibrosis by administering the composition according to the present disclosure.

As used herein, the term “treatment” refers to all activities that improve or beneficially change the symptoms of pulmonary fibrosis by administering the composition according to the present disclosure.

As used herein, the term “subject” refers to a subject in need of a method for preventing or treating diseases, and more specifically, a primate, such as a human or non-human, and a mammal, such as a mouse, a rat, a dog, a cat, a horse, a cow, and the like.

Hereinafter, preferred embodiments of the present disclosure will be presented to aid understanding of the present disclosure. However, the following examples are only provided to more easily understand the present disclosure, and the content of the present disclosure is not limited by the following examples.

### EXAMPLE

### Example 1. Experiment Preparation and Experiment Method

1-1. Cell Culture

An epithelial cell line, Beas-2B cells, was cultured in RPMI (Invitrogen) medium supplemented with 10% fetal bovine serum (FBS).

1-2. Transfection

Vector and siRNA were prepared using lipofectamin 2000 (Invitrogen) according to the provided manual.

1-3. Western Blot

Protein was separated from the cells using a lysis buffer [40 mM Tris-HCl (pH 8.0), 120 mM NaCl, and 0.1% Nonidet-P40] to which a protease inhibitor is added, and then transferred to SDS-PAGE and nitrocellulose membrane (Amersham, Arlington Heights, IL). The membrane was blocked with 5% non-fat dry milk (in Tris-buffered saline) and then underwent a reaction with a primary antibody at a temperature of 4° C. The membrane was reacted with peroxidase-conjugated a secondary antibody and then visualized using enhanced chemiluminescence (ECL, Amersham, Arlington, Heights, IL).

1-4. Real-Time Quantitative PCR

RNA was extracted using triazol (Invitrogen, Carlsbad, CA, USA). qRT-PCR was performed using SensiFAST™ SYBR No-ROX Kit (Bioline Reagents, UK), and the reaction was performed using Rotor-Gene Q (Qiagen, Seoul, Korea). Results were calculated using the ΔΔCt method and normalized to β-actin.

1-5. Immunohistochemistry (IHC)

Mouse tissue was fixed in formalin to produce a paraffin block. The paraffin-embedded tissue was cut, and the paraffin was removed using xylene and 100%, 95%, 80%, and 70% ethanol. The cut tissue was subjected to hematoxylin & eosin (H&E) staining, Sirius red staining (abc150681), and DAB staining.

In the process of DAB staining, the primary antibody underwent reactions overnight at a temperature of 4° C. and then underwent reactions with a biotinylated secondary antibody and an ABC reagent (Vector Laboratories, USA) for 1 hour each. For a color reaction, 3,3-diaminobenzidine (Vector Laboratories) was used, and contrast staining was performed using hematoxylin. Thereafter, the tissue underwent reactions with 70%, 80%, 95%, and 100% ethanol and xylene, and then was mounted with a Canada balsam mounting medium. Images were observed using a DP71 system of an IX71 microscope (Olympus, Seoul, Korea).

1-6. Human Tissue Microarray

Microarray samples of human pulmonary interstitial fibrosis tissue were purchased from US-Biomax (LC561), and expression of CSF3 was analyzed by IHC.

1-7. Data Analysis

Gene expression omnibus (GEO) datasets (GSE10667, GSE71351, GSE134692) were used for genome analysis of pulmonary fibrosis patients. Gene set enrichment analysis (GSEA) was performed based on the Molecular Signature Database (MsigDB).

1-8. Animal Experiments

To prepare a pulmonary fibrosis model, 100 mg/kg of bleomycin sulfate was intraperitoneally injected into male C57BL/6 mice four times (day 1, day 4, day 7, and day 10). 250 μg/kg of a neutralizing antibody was intraperitoneally injected four times (day 12, day 14, day 16, and day 18) after pulmonary fibrosis was induced. On day 21, the mice were sacrificed, and the lung tissue was extracted.

1-9. Hydroxyproline Assay

Hydroxyproline levels were assayed in a mouse tissue lysate using a hydroxyproline assay kit (ab222941, abcam, UK). The experiment was performed based on the manufacturer instructions.

1-10. Co-Immunoprecipitation

The cell protein extracted with the lysis buffer underwent reactions with the primary antibody overnight at a temperature of 4° C., and then precipitated using protein A-agarose beads (Santa Cruz Biotechnology, Inc.). The protein A-agarose beads were washed with cold PBS, and the precipitated protein was analyzed by Western blotting.

1-11. In Situ Proximity Ligation Assay (PLA)

The cells cultured on a coverslip were fixed with 4% paraformaldehyde and then permeabilized with a PBS solution containing 0.1% Triton X-100 and 10% fetal bovine serum. A CSF3 antibody and a STAT3 antibody were used as primary antibodies and underwent reactions overnight. Then, PLA was performed using a Duolink Detection Kit (Sigma) product. A confocal microscopy was used for visualization.

### Example 2. Analysis of CSF3 Expression in Idiopathic Pulmonary Fibrosis Patient Tissue and Mouse Model

2-1. Confirmation of CSF3 Expression

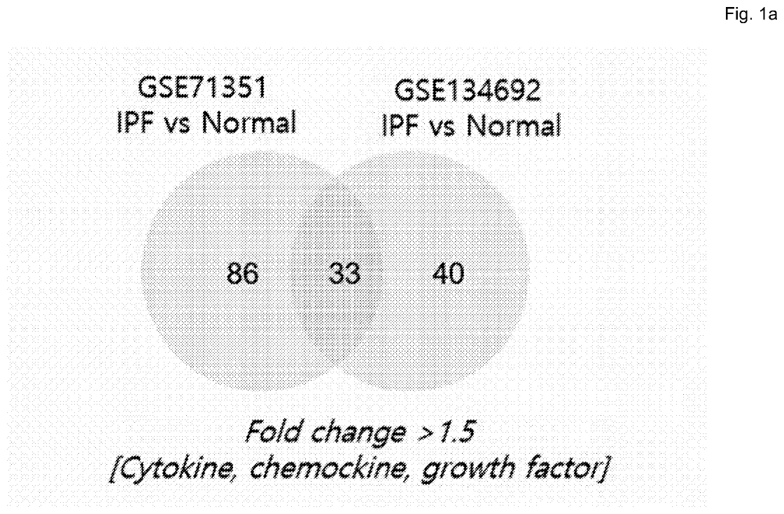

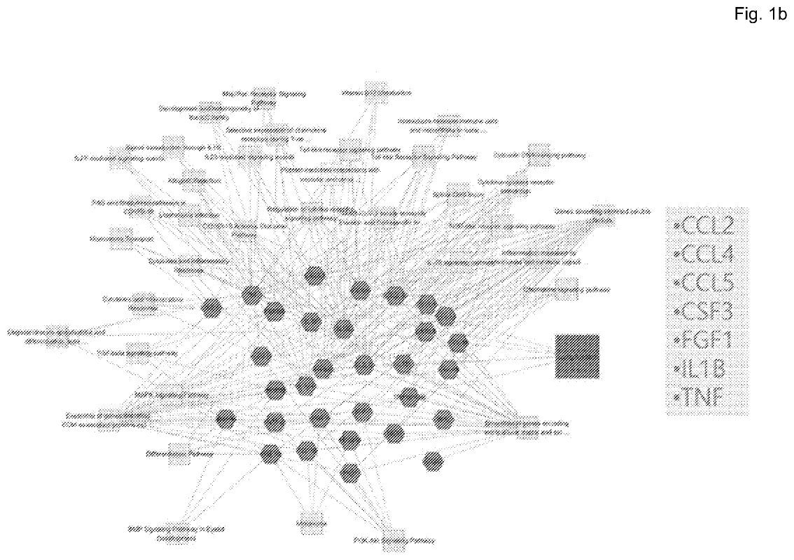

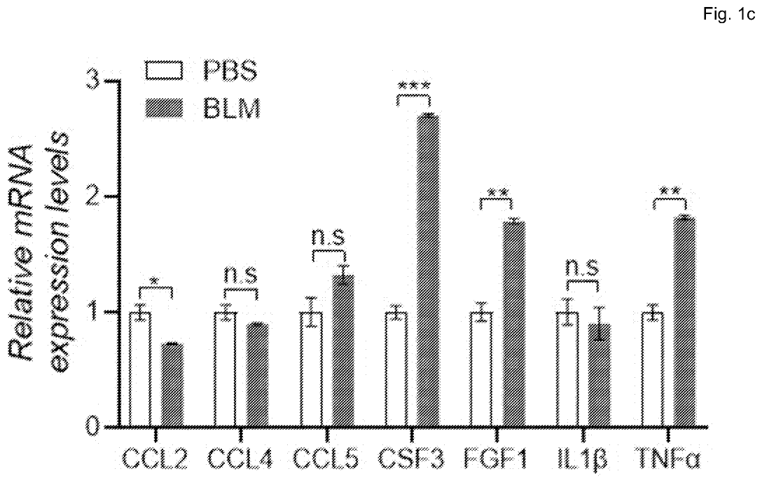

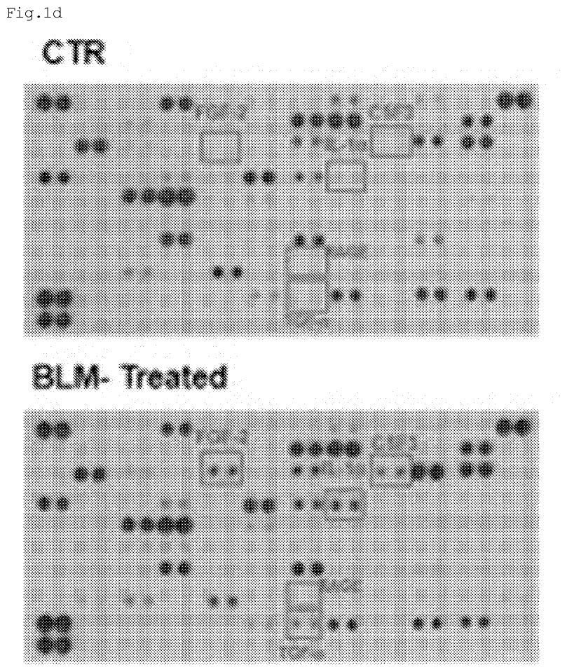

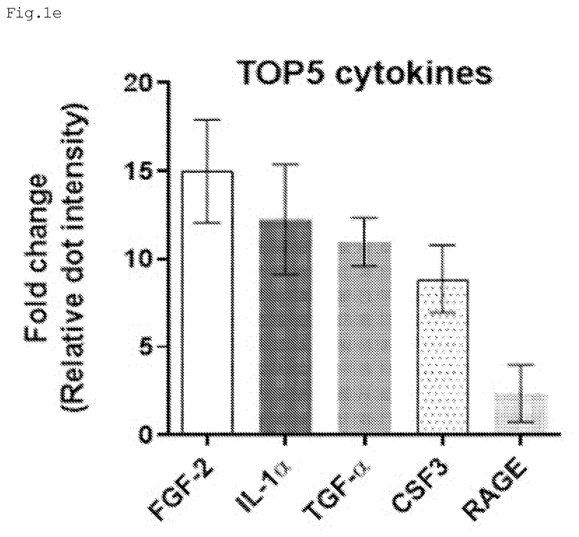

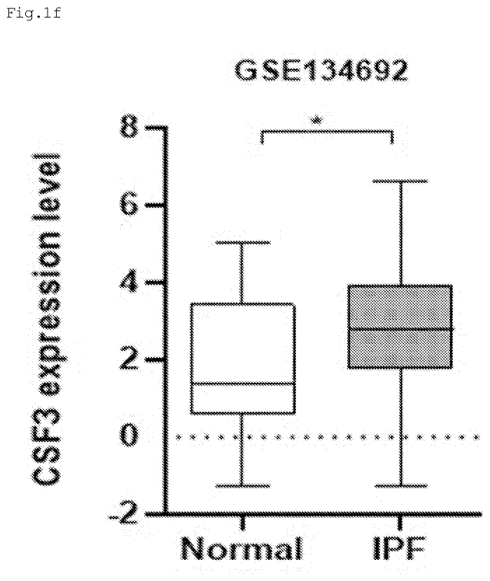

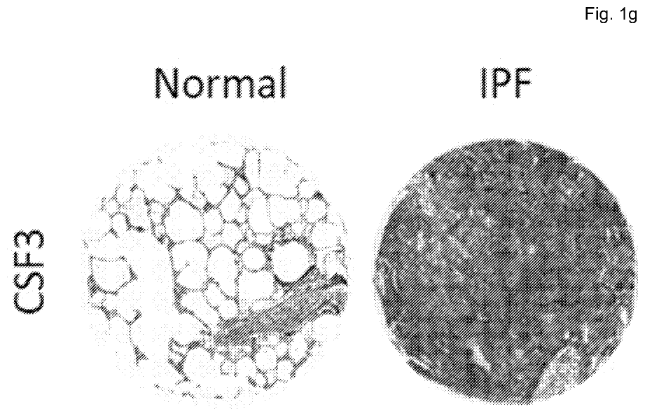

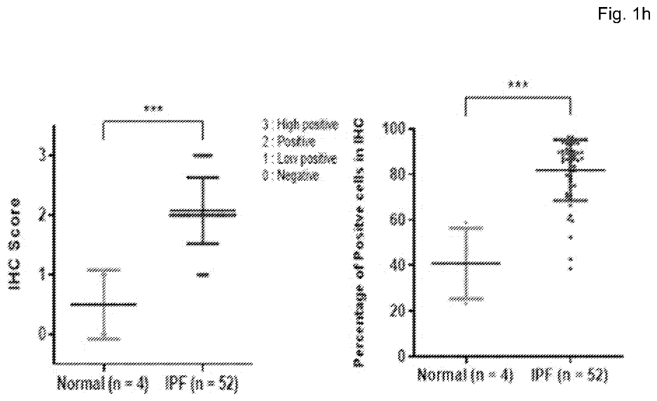

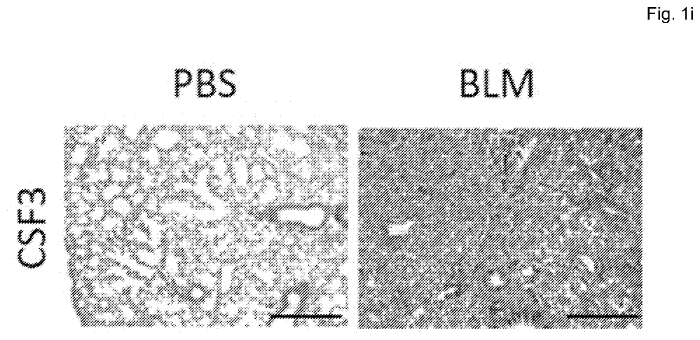

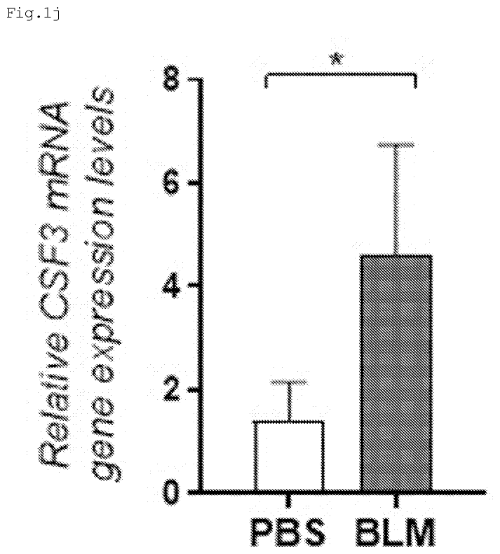

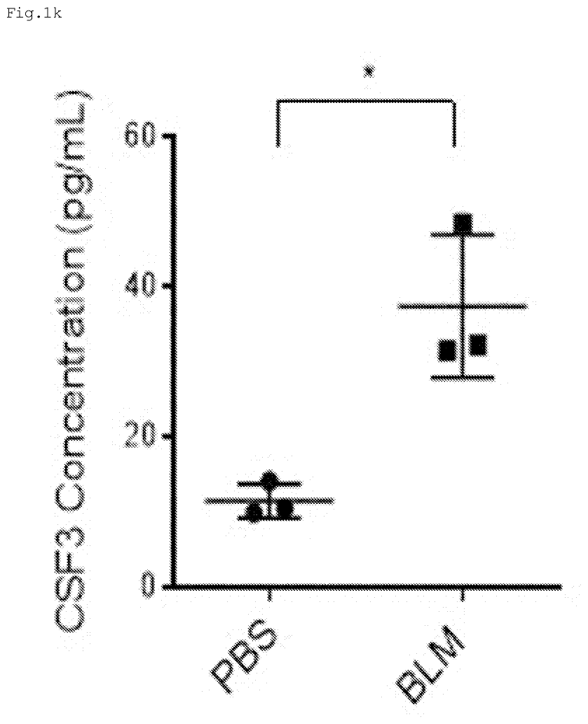

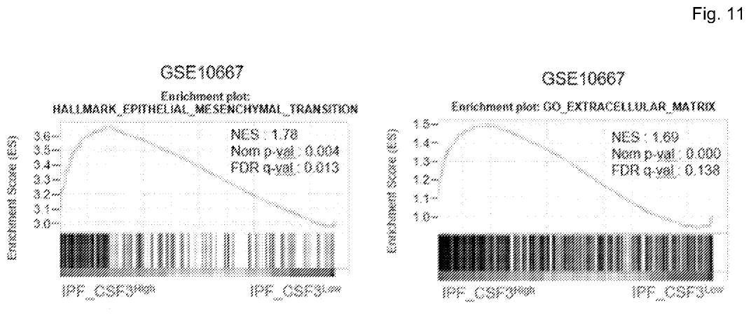

The GEO database was used to discover novel therapeutic targets for pulmonary fibrosis. The expression of secretion factors confirmed to be higher in the lung tissue of pulmonary fibrosis patients than in the lung tissue of normal patients was analyzed. As a result, 33 types of secretion factors with commonly increased expression were identified in the pulmonary fibrosis patient dataset (FIG. 1A). As a result of performing cytoscape analysis on the 33 types of factors, 7 final candidates (CCL2, CCL4, CCL5, CSF3, FGF1, IL1B, and TNF) were selected (FIG. 1B). As a result of confirming the expression levels of the selected seven secretion factors in a lung epithelial cell line, it was confirmed that the expression of CSF3 was increased the most by BLM treatment.

The increase in the CSF3 expression was similarly found when performing cytokine array analysis on Beas-2b cells and a BLM-treated group to discover cytokines capable of inducing EMT of lung epithelial cell (FIGS. 1D and 1E). In addition, CSF3 secretion was significantly increased in idiopathic pulmonary fibrosis (IPF) patients (FIG. 1F), so the inventors of the present disclosure identified CSF3 as a meaningfully novel target for pulmonary fibrosis. As a result of performing immunohistochemical staining on the IPF patient tissue microarrays, it was confirmed that the CSF3 expression was higher in the lung tissue of the patients than in normal tissue (FIGS. 1G and 1H). Even when performing immunohistochemical staining (FIG. 1I), RT-qPCR (FIG. 1J), and ELISA (FIG. 1K) on a Bleomycin (BLM)-induced idiopathic pulmonary fibrosis mouse model, similar results were obtained.

In addition, when performing Gene Set Enrichment Analysis (GSEA) on the idiopathic pulmonary fibrosis patients with high CSF3 expression, it was confirmed that the genes related to extracellular matrix (ECM) and epithelial-mesenchymal transition (hereinafter, referred to as EMT) were highly expressed.

Through the above experiments, the inventors of the present disclosure confirmed that the CSF3 expression was high in the pulmonary fibrosis patients and the pulmonary fibrosis mouse model, thereby discovering CSF3 as a new treatment target for pulmonary fibrosis and confirming its correlation with EMT.

2-2. Confirmation of CSF Family Expression

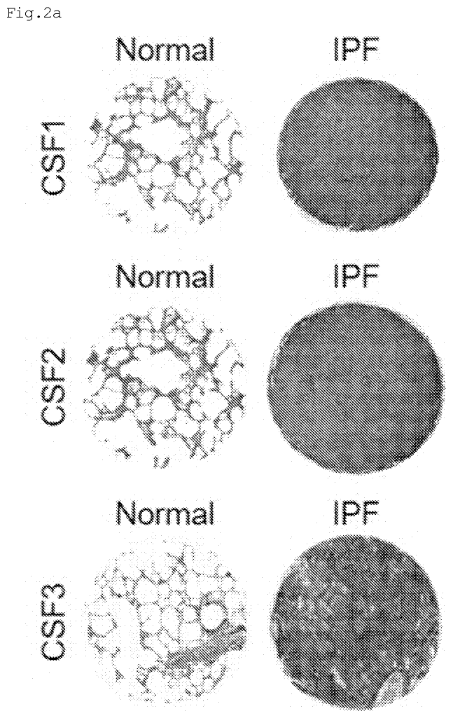

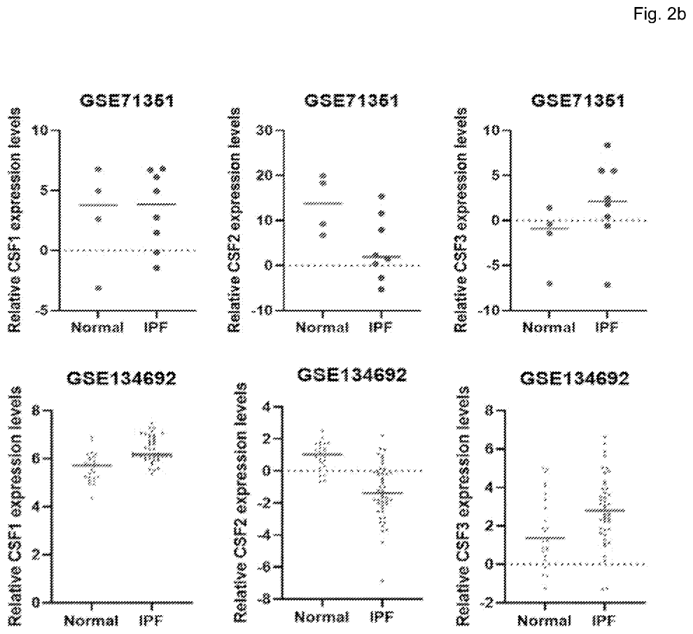

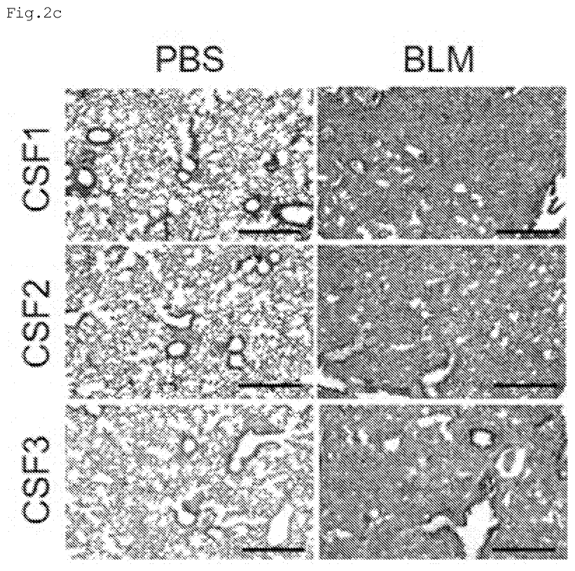

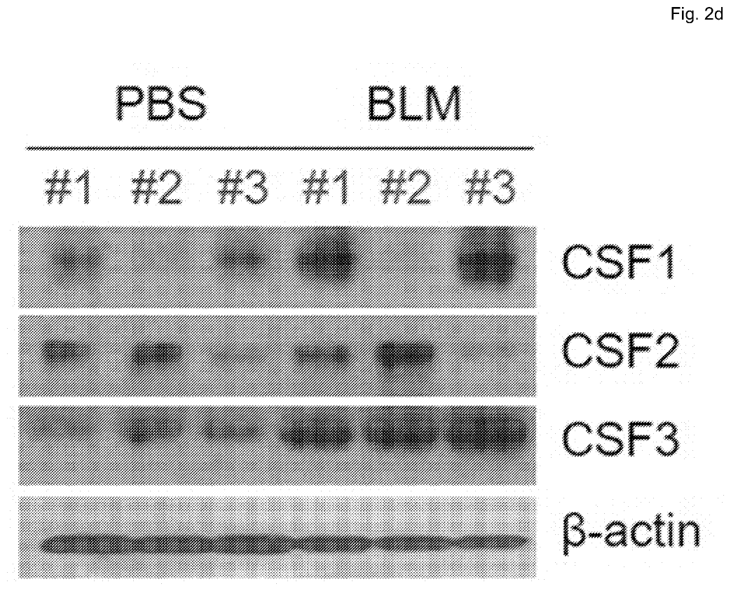

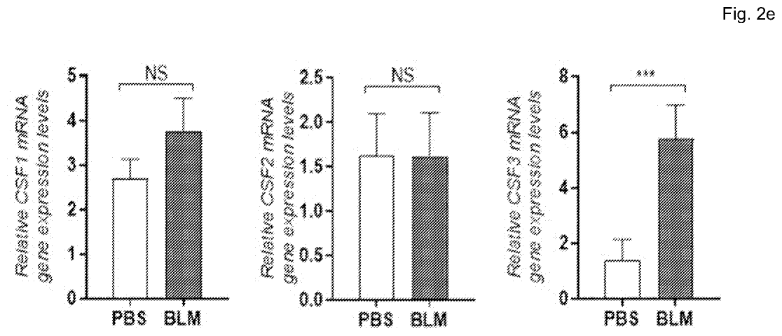

In Example 2-1, while CSF3 was confirmed to be able to be used as the target for pulmonary fibrosis, the expression of CSF1 and CSF2, members of the CSF3 family, were examined to confirm whether CSF1 and CSF2 were remarkably expressed and able to be used as targets for pulmonary fibrosis as in CSF3. To compare the expression patterns of the CSF family, immunohistochemical staining was performed on the tissue microarrays of the IPF patients. As a result, it was confirmed that only CSF3 was more highly expressed in the patient tissue than in the normal tissue (FIG. 2A). In addition, when analyzing the GEO database, it was confirmed that, among the three genes belonging to the CSF family, only CSF3 was highly expressed in the IPF patients (FIG. 2B). Likewise, even when performing immunohistochemical staining (FIG. 1C), Western blotting (FIG. 1D), and RT-qPCR (FIG. 1E) on the bleomycin-induced mouse model to confirm the expression of the CSF3 family, the expression of CSF1 and CSF2 in the pulmonary fibrosis lung tissue were similar to that of in the normal tissue. However, it was confirmed that CSF3 expression was significantly increased in the pulmonary fibrosis lung tissue.

Through the above results, the inventors of the present disclosure confirmed that, among the CSF family, only CSF3 was highly expressed in the pulmonary fibrosis patients and pulmonary fibrosis mouse model.

### Example 3. Induction of EMT and Pulmonary Fibrosis in Lung Tissue by CSF3

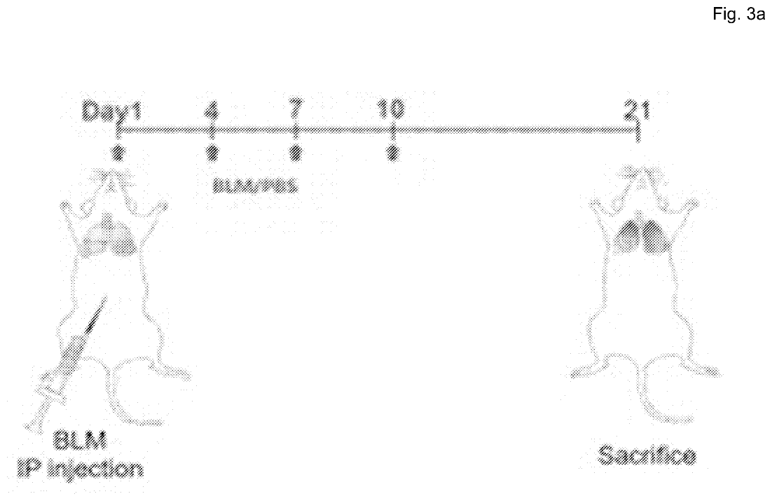

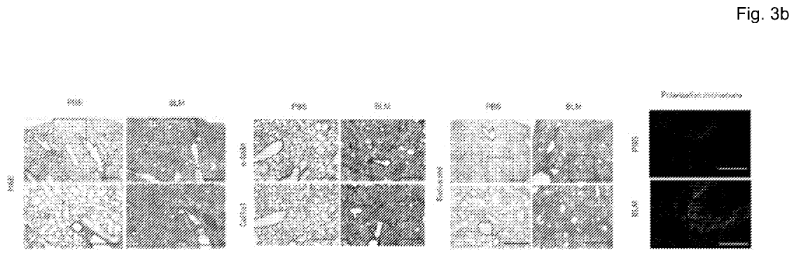

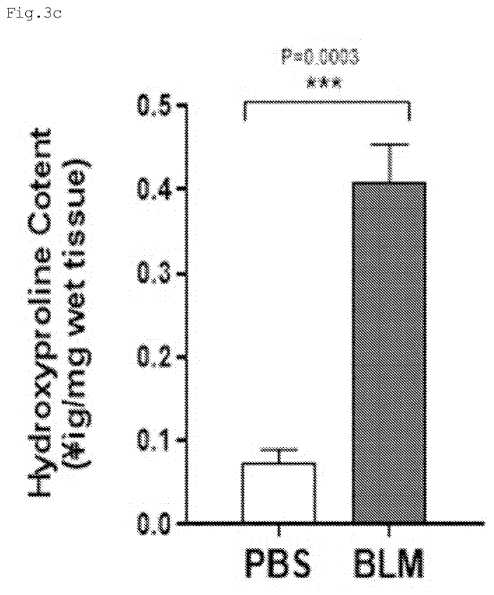

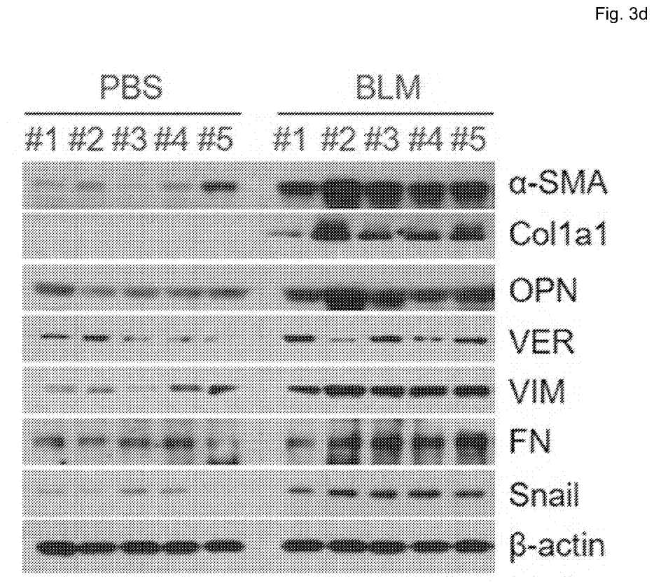

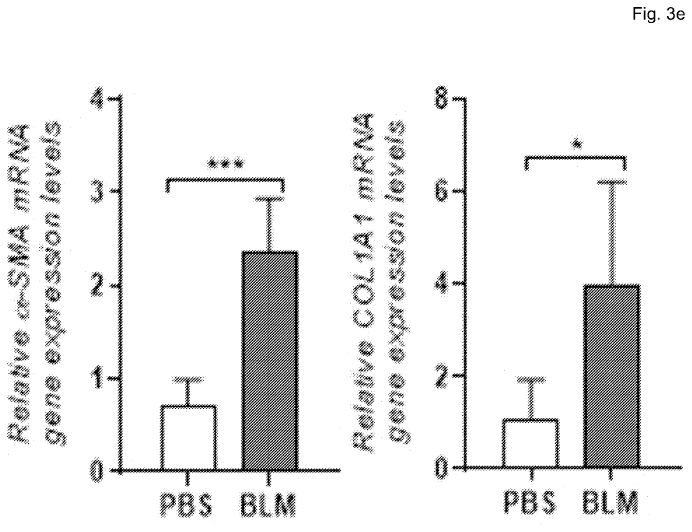

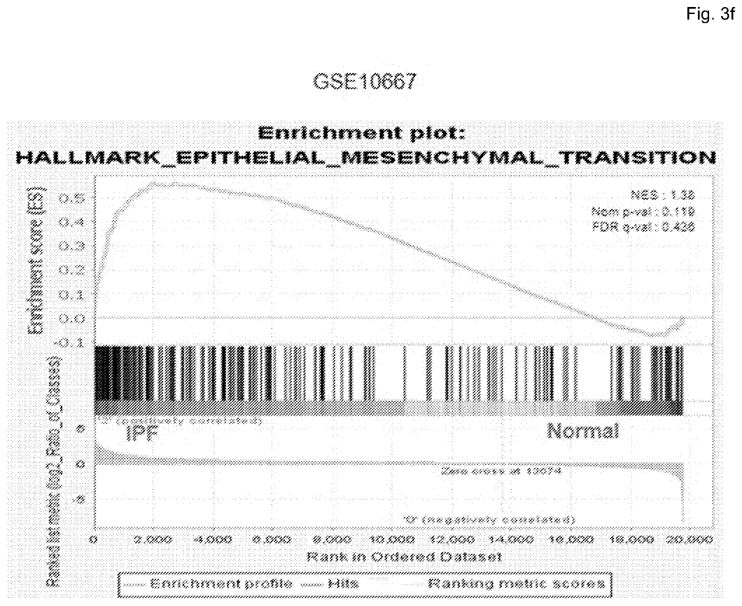

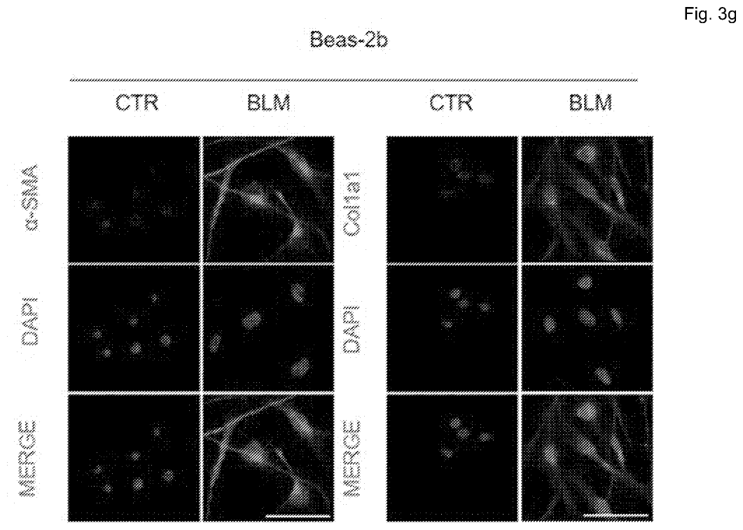

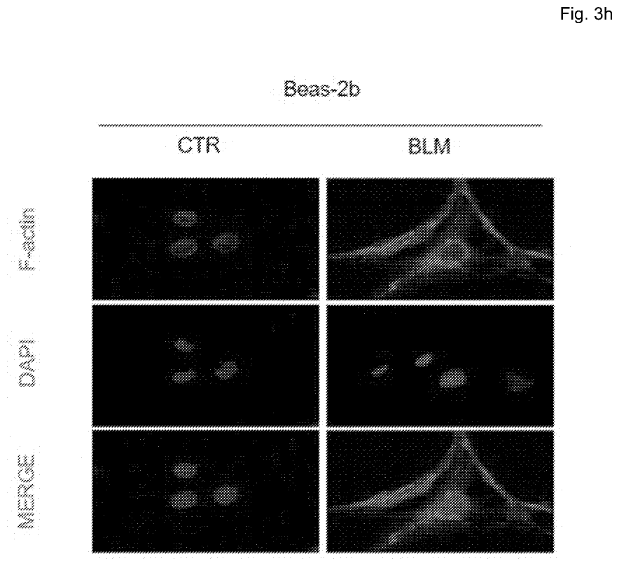

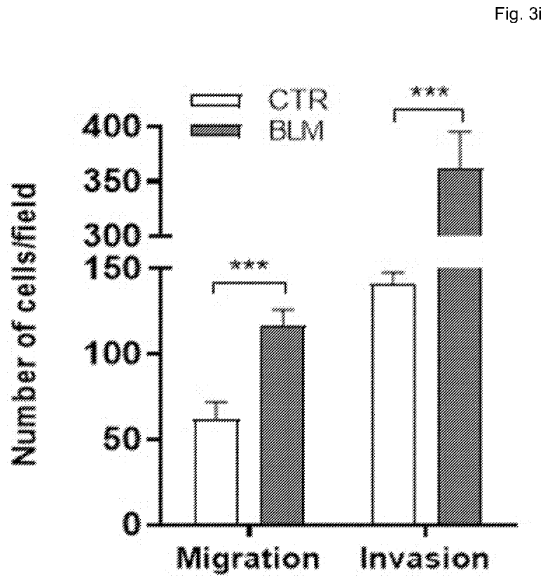

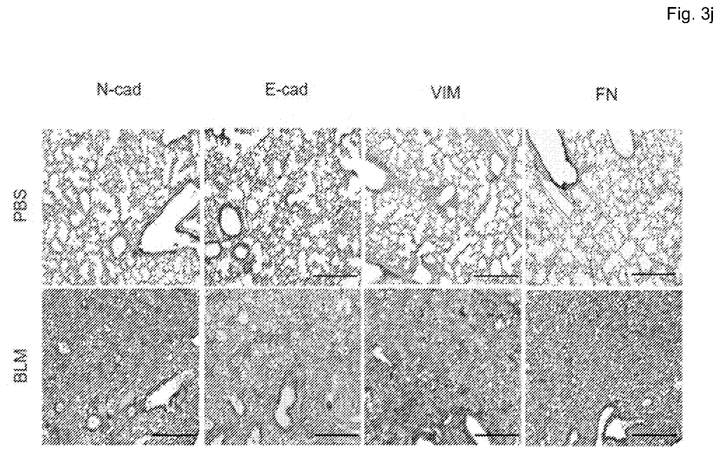

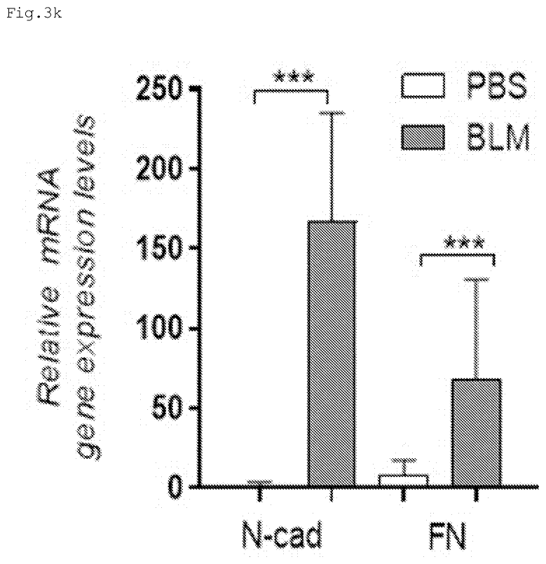

\textcircled{1} To construct a mouse model in which idiopathic pulmonary fibrosis was induced (hereinafter, referred to as BLM-induced idiopathic pulmonary fibrosis mouse model), bleomycin was intraperitoneally administered to mice (FIG. 3A). When observing lung tissue of the mouse model through immunohistochemical staining (FIG. 3B), hydroxyproline analysis (FIG. 3C), Western blotting (FIG. 3D), and q-RT PCR (FIG. 3E), it was confirmed that α-SMA and COL1A1, markers of lung tissue fibrosis and idiopathic pulmonary fibrosis, were increased. Thus, the BLM-induced idiopathic pulmonary fibrosis mouse model was established. As a result of analyzing the lung tissue of idiopathic pulmonary fibrosis and normal tissue by performing Gene Set Enrichment Analysis (GSEA) (FIG. 3F), signature genes of EMT were highly expressed in the lung tissue of idiopathic pulmonary fibrosis.

In addition, when treating a lung epithelia cell line Beas-2b with bleomycin and performing immunohistochemical staining for confirmation, the expression of α-SMA and COL1A1 was increased (FIG. 3G), the Beas2-b cells changed into fusiform cells (FIG. 3H), and mobility of the Beas2-b cells was also increased (FIG. 3I).

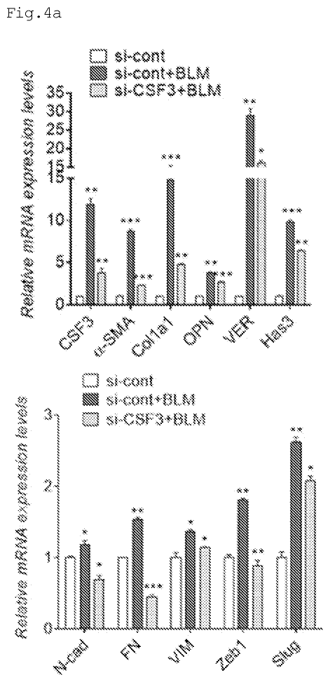

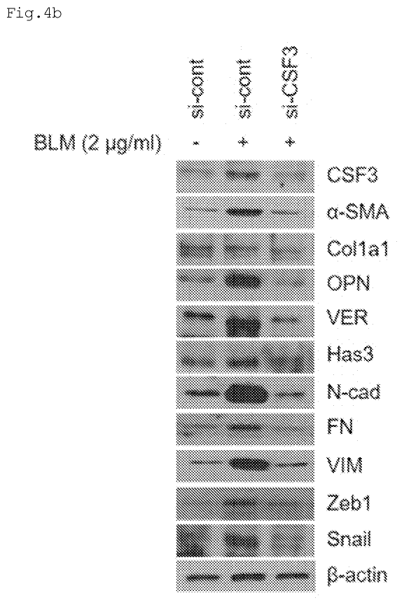

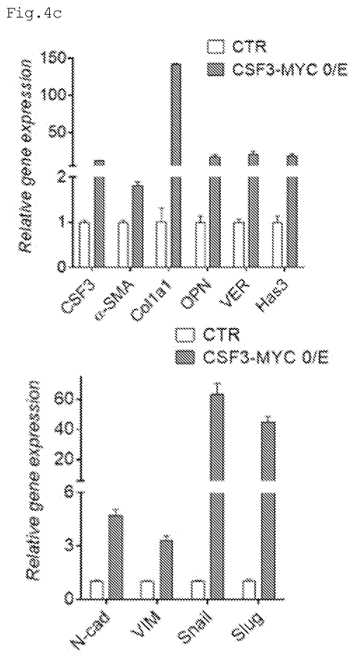

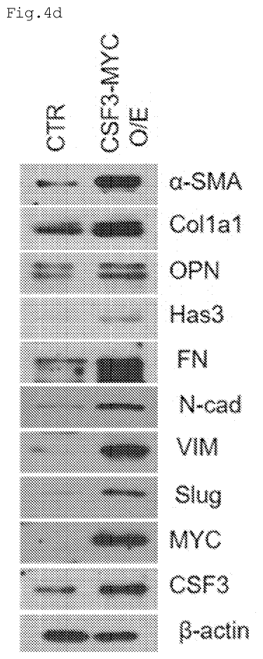

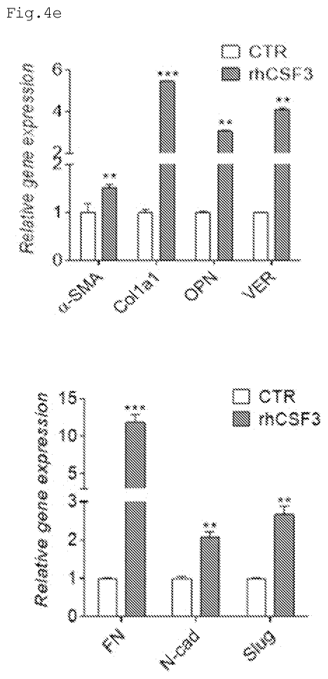

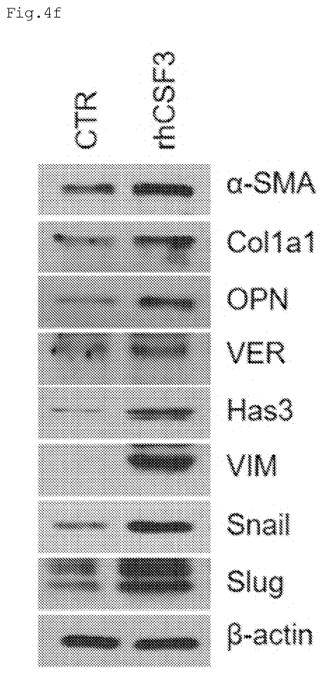

When performing immunohistochemical staining (FIG. 3J) and RT-qPCR (FIG. 3K) on the BLM-induced idiopathic pulmonary fibrosis mouse model for observation, it was confirmed that the expression of the pulmonary fibrosis markers as well as EMT markers, N-cad, fibronectin (FN), and vimentin (VIM), was increased.
\textcircled{2} To confirm whether EMT and differentiation into myofibroblasts are induced by CSF3 in the lung epithelial cell line Beas-2b, the bleomycin-treated lung epithelial cell line was treated with si-CSF3 and then observed by performing RT-qPCR (FIG. 4A) and Western blotting (FIG. 4B). As a result, it was confirmed that when CSF3 expression was inhibited by siRNA treatment, the EMT markers induced by bleomycin were decreased once more.

Even in the case of the lung epithelial cell line Beas-2b overexpressing CSF3, when performing RT-qPCR (FIG. 4C) and Western blotting (FIG. 4D), it was confirmed that the EMT markers were increased. Such results were confirmed to be similar to the increased levels of the EMT markers when treating the Beas-2b with recombinant human-CSF3 (rhCSF3) and then performing RT-qPCR (FIG. 4E) and Western blotting (FIG. 4F).

Through the above results, the inventors of the present disclosure confirmed that the EMT levels were also increased in the idiopathic pulmonary fibrosis patients and the BLM-induced idiopathic pulmonary fibrosis mouse model.

### Example 4. Identification of Epithelial-Mesenchymal Transition (EMT) Regulatory Mechanism by CSF3

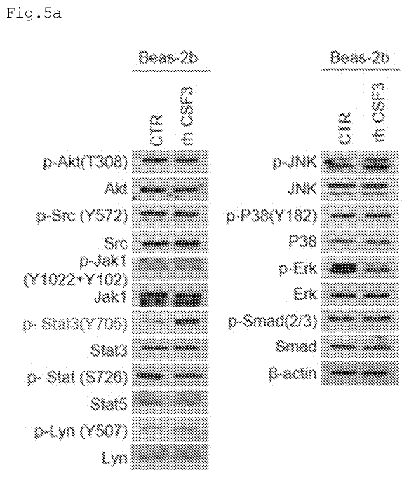

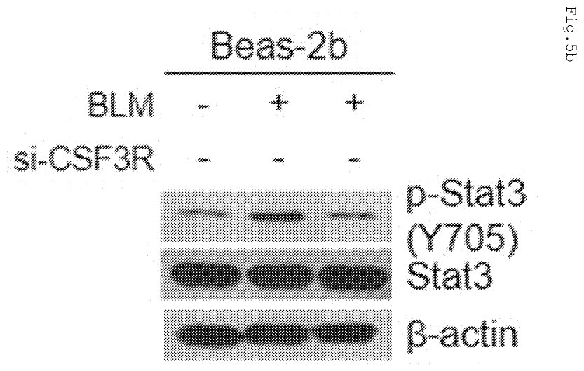

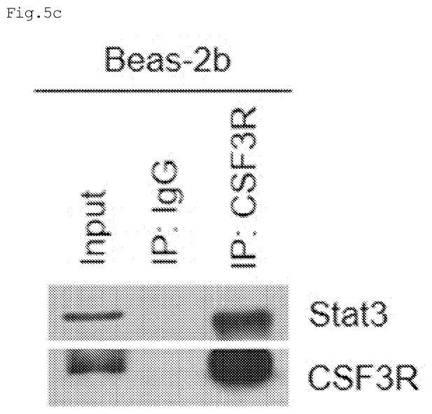

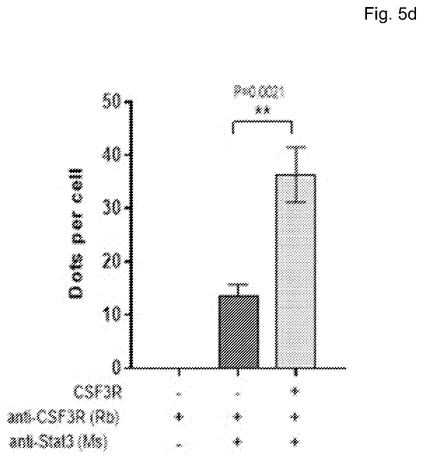

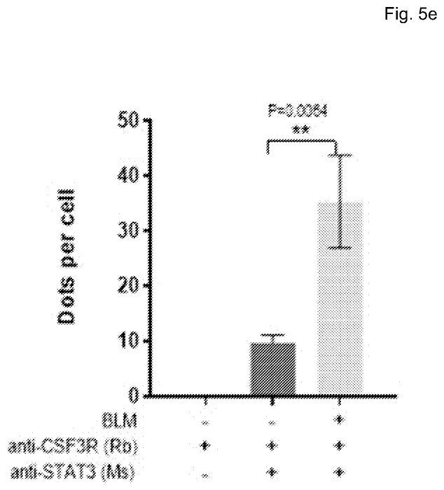

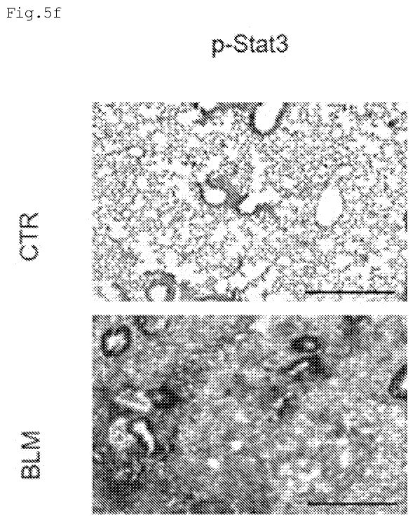

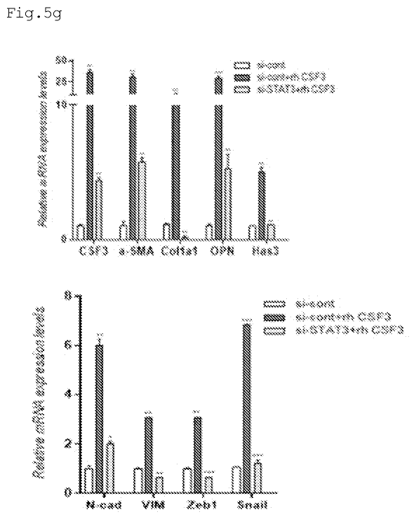

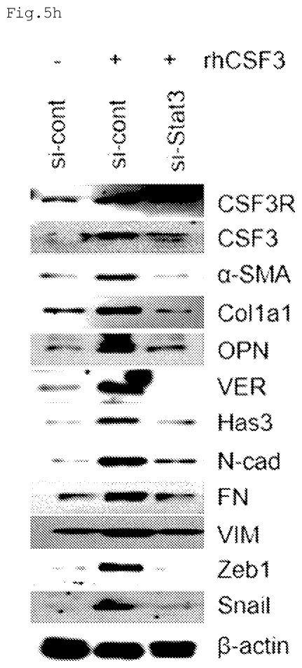

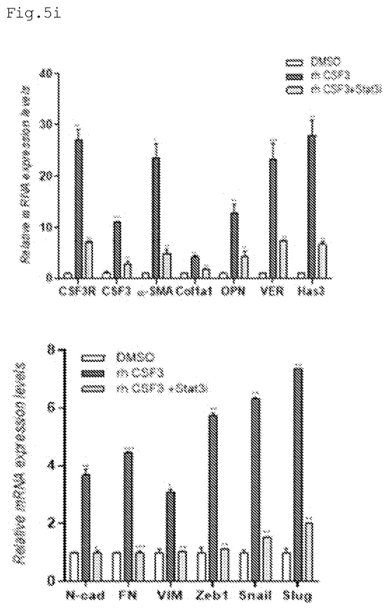

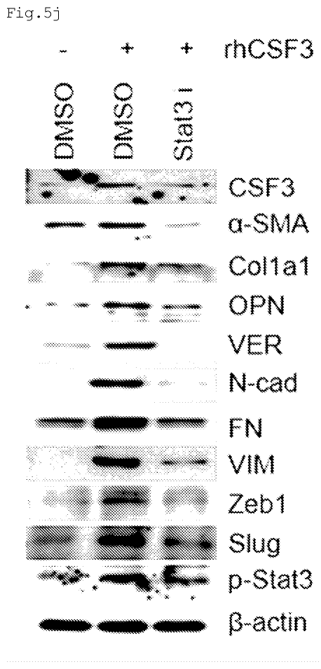

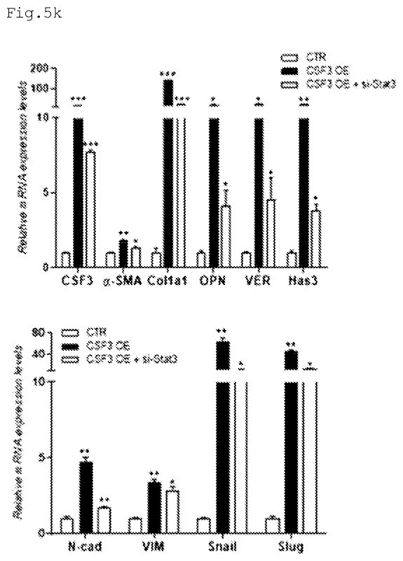

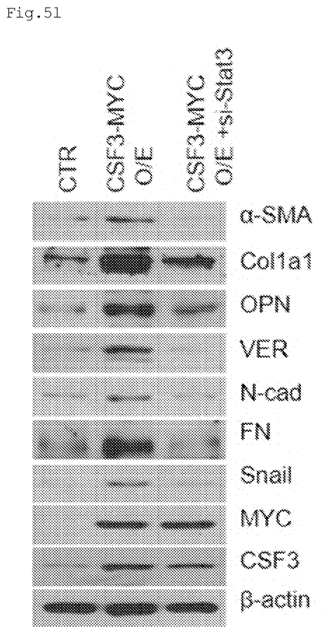

In Example 3, it was confirmed that CSF3, a promising marker for idiopathic pulmonary fibrosis discovered in the present disclosure, was able to induce idiopathic fibrosis and EMT. To identify EMT-induced downstream signaling mechanism by CSF3 in pulmonary epithelial cells, a lung epithelial cell line Beas-2b was treated with rh-CSF3, and then screening was performed (FIG. 5A). As a result, it was confirmed that STAT3 activity was greatly increased, and through Western blotting it was confirmed that STAT3 activity induced by bleomycin was decreased as the expression of CSF3 receptor (CSF3R) was inhibited. In addition, through Co-Immunoprecipitation (Co-IP) (FIG. 5C) and in situ PLA (FIGS. 5D and 5E), it was confirmed that STAT3 was directly bound to CSF3R.

It was confirmed that even the p-STAT3 protein was more highly expressed in lung tissue of a BLM-induced idiopathic pulmonary fibrosis mouse model than that of a control group. In addition, it was confirmed by RT-qPCR (FIGS. 5G, 5I, and 5K) and Western blotting (FIGS. 5H, 5J, and 5L) that the expression of EMT and ECM components induced by rhCSF3 treatment or CSF3 overexpression in the lung epithelial cell line Beas-2b were decreased by si-STAT3 and STAT3 inhibitors.

Through the above experimental results, the inventors of the present disclosure confirmed that STAT3 mediated CSF3-induced EMT of the lung epithelial cell and transdifferentiation of the lung epithelial cell into myofibroblasts.

### Example 5. Preventive Effect of Pulmonary Fibrosis by CSF3 Neutralizing Antibody Treatment

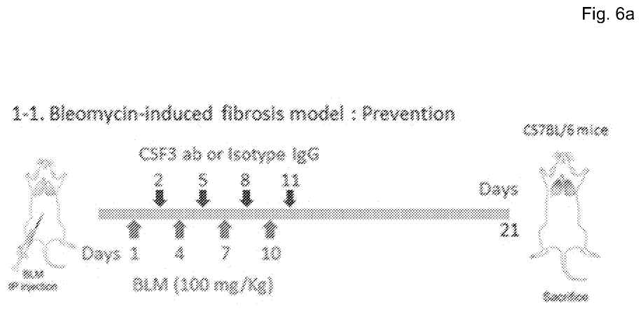

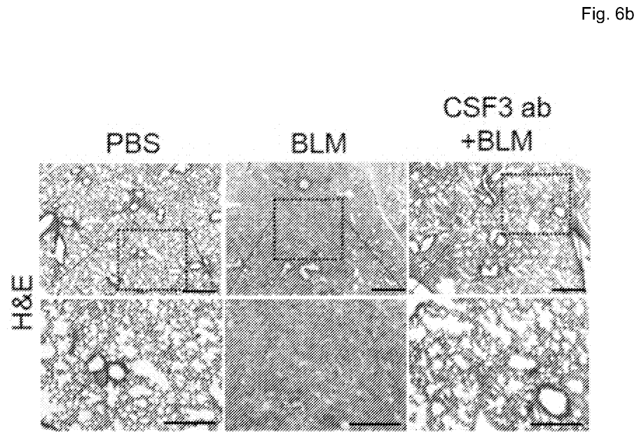

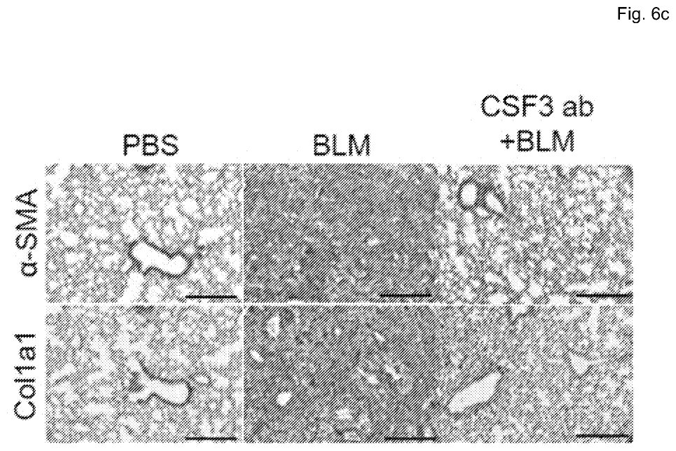

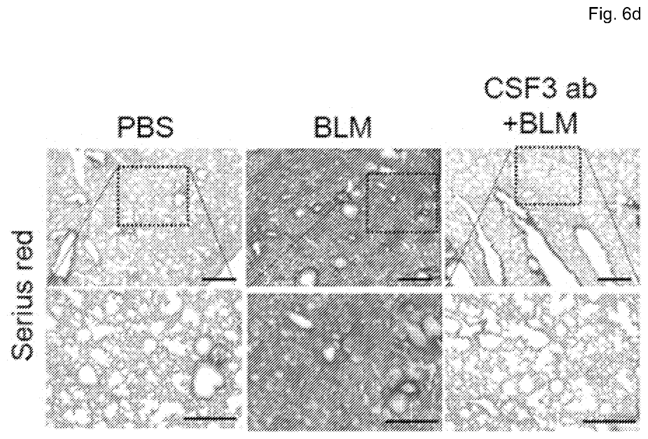

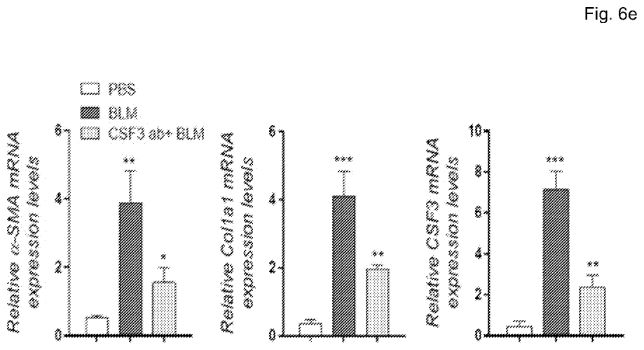

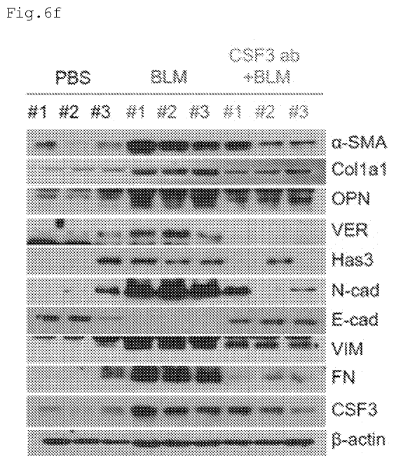

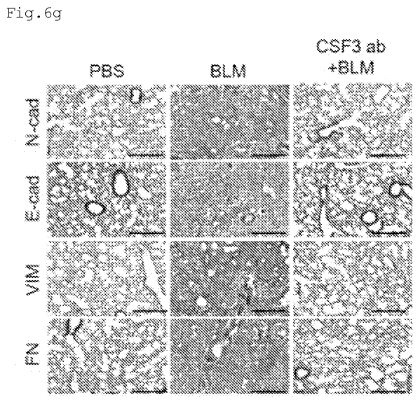

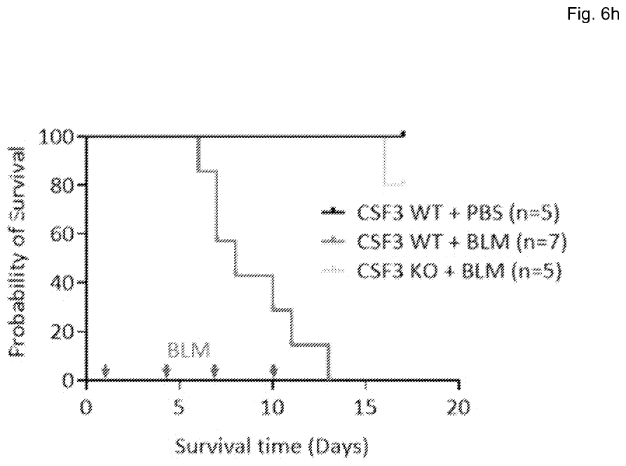

To confirm whether an effect of preventing pulmonary fibrosis was obtained when blocking CSF3 in advance, bleomycin and a neutralizing antibody that inhibited CSF3 activity (hereinafter, referred to as anti-CSF3 antibody) were treated in combination (FIG. 6A). When pre-blocking CSF3 by pretreating a BLM-induced idiopathic pulmonary fibrosis mouse model with the anti-CSF3 antibody, and then performing H&E staining (FIG. 6B), immunohistochemical staining (FIG. 6C), and Sirius red staining (FIG. 6D), it was confirmed that the occurrence of idiopathic pulmonary fibrosis was decreased. In the case of inhibiting CSF3 of the present disclosure, it was confirmed that the effect of preventing idiopathic pulmonary fibrosis was obtained. When performing RT-qPCR (FIG. 6E), Western blotting (FIG. 6F), and immunohistochemical staining (FIG. 6G) on the BLM-induced idiopathic pulmonary fibrosis mouse model pretreated with the anti-CSF3 antibody, it was confirmed that pulmonary fibrosis marker protein and EMT marker protein failed to be induced.

In addition, when comparing C57BL/6 control mice and CSF3-deficient mice by treating each group with bleomycin, it was confirmed that the survival rate of the CSF3 wild-type mouse group was lower than that of the CSF3-deficient mouse group.

Through the above results, the inventors of the present disclosure confirmed that the occurrence of idiopathic pulmonary fibrosis was able to be prevented when pretreated with the neutralizing antibody capable of inhibiting CSF3 activity.

### Example 6. Therapeutic Effect of CSF3 Neutralizing Antibody Treatment on Pulmonary Fibrosis

6-1. Confirmation of Therapeutic Effect of CSF3 Neutralizing Antibody

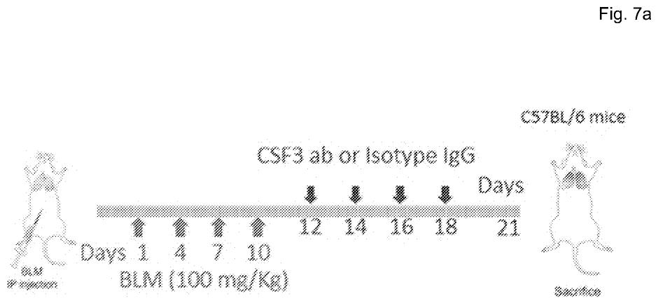

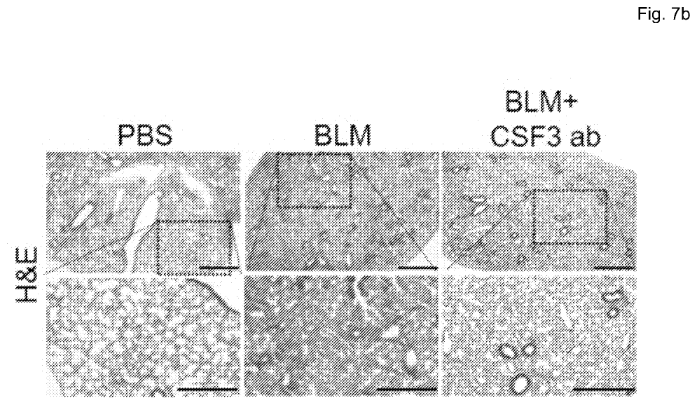

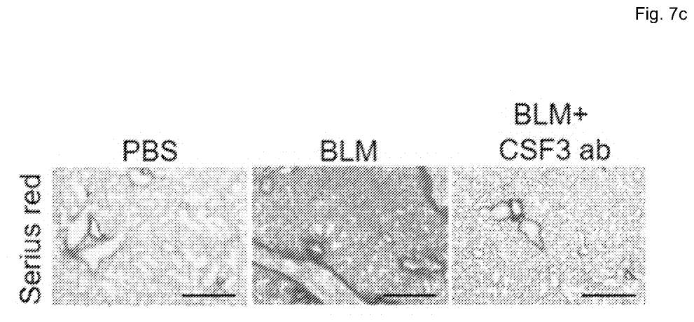

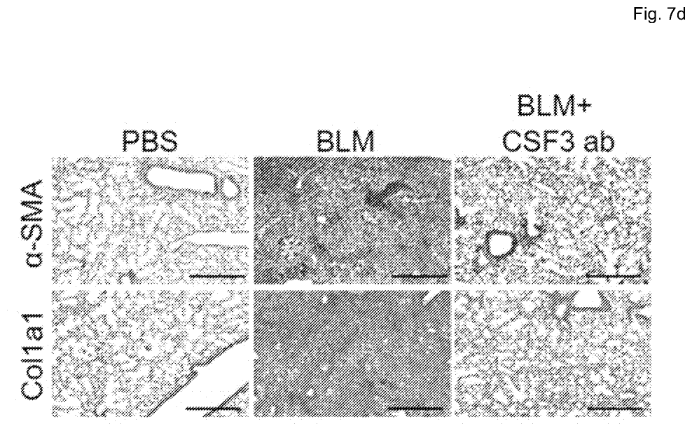

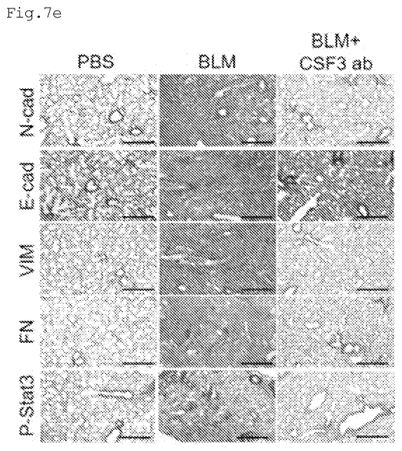

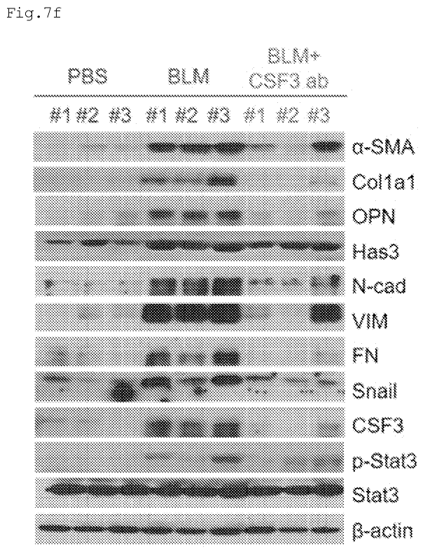

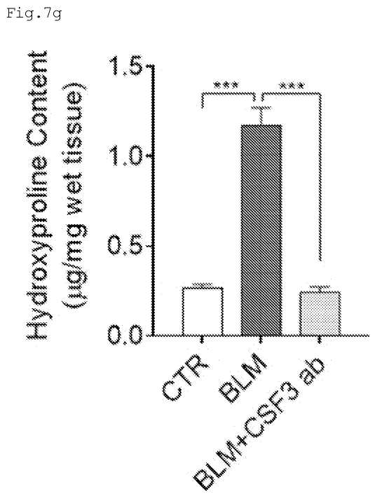

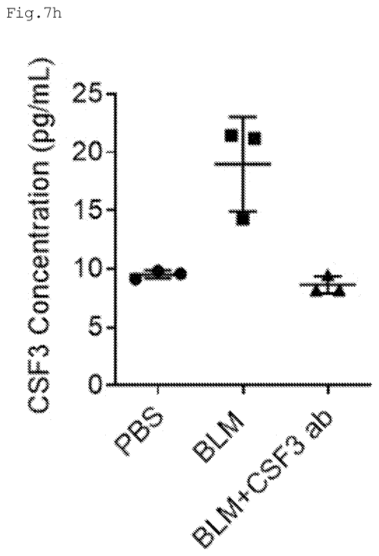

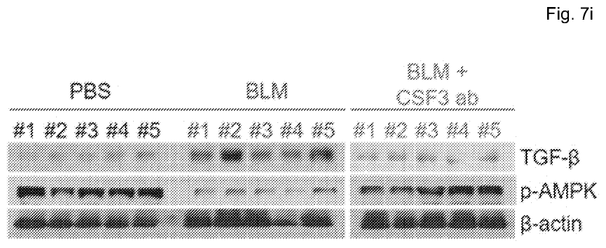

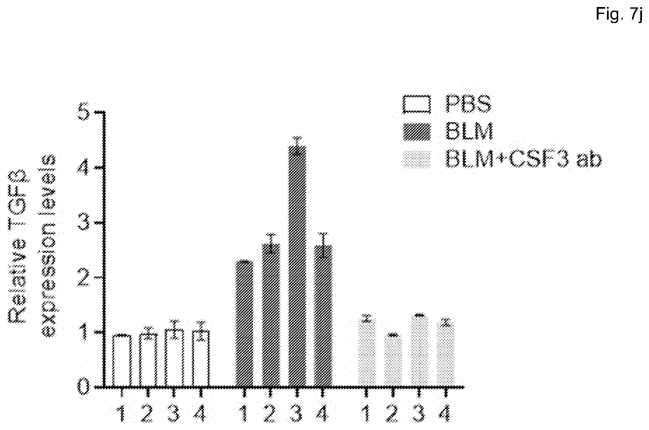

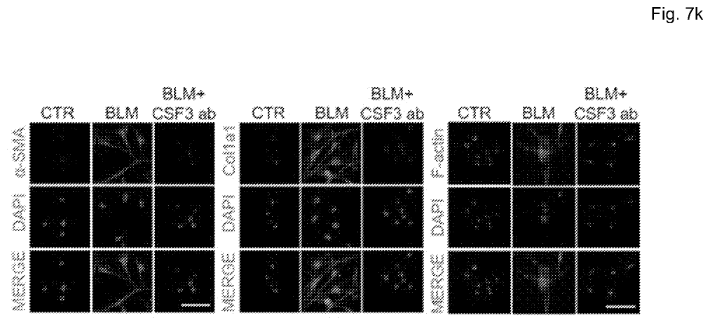

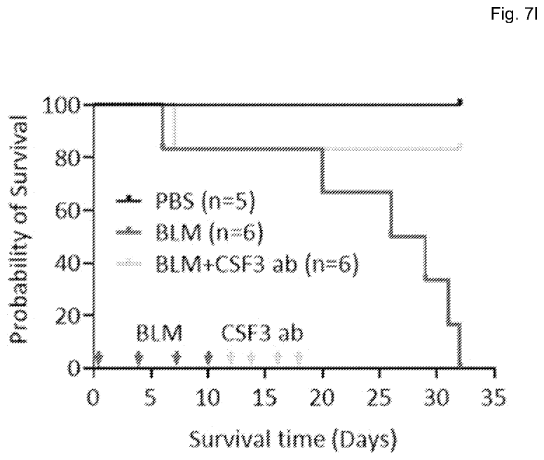

To confirm an effect of treating pulmonary fibrosis by inhibiting CSF3, Anti-CSF3 neutralizing antibody was administered three times to a BLM-induced idiopathic pulmonary fibrosis mouse model (FIG. 7A). As a result of observation through H&E staining (FIG. 7B), Sirius red staining (FIG. 7C), and immunohistochemical staining (FIG. 7D), it was confirmed that idiopathic pulmonary fibrosis symptoms were relieved. As a result of observation through immunohistochemical staining (FIG. 7E) and Western blotting (FIG. 7F) in a mouse group administered with the anti-CSF3 neutralizing antibody, it was confirmed that EMT marker protein was also significantly reduced. Even when performing hydroxyproline assay on an experimental group treated with the anti-CSF3 neutralizing antibody, it was confirmed that the increased hydroxyproline in lung tissue where pulmonary fibrosis was induced significantly decreased (FIG. 7G).

When treating the BLM-induced idiopathic pulmonary fibrosis mouse model with the anti-CSF3 neutralizing antibody of the present disclosure, it was confirmed by ELISA that the increased CSF3 secretion in the lung tissue of the mouse model was reduced again in the group treated with the anti-CSF3 neutralizing antibody (FIG. 7H). In addition, in the case of the BLM-induced idiopathic pulmonary fibrosis mouse model, even when performing Western blotting (FIG. 7I) and RT-qPCR (FIG. 7J) for p-AMPK and TGF-β, it was confirmed that TGF-β expression, reported to be increased in pulmonary fibrosis, increased while AMPK activity was decreased. However, when administering the anti-CSF3 neutralizing antibody to the BLM-induced idiopathic pulmonary fibrosis mouse model, it was confirmed that the TGF-β expression and AMPK activity were restored to a level similar to that of normal tissue.

When treating the anti-CSF3 neutralizing antibody, it was confirmed by immunohistochemical staining that pulmonary fibrosis markers, which was increased in bleomycin-treated Beas-2b, were decreased (FIG. 7k). In addition, it was confirmed that the survival rate of the BLM-induced idiopathic pulmonary fibrosis mouse model was significantly increased in the group treated with the anti-CSF3 neutralizing antibody.

Through the above results, the inventors of the present disclosure specifically confirmed the fact that the anti-CSF3 neutralizing antibody of the present disclosure had the therapeutic effect on pulmonary fibrosis.

6-2. Confirmation of Therapeutic Effect of CSF3 Family Neutralizing Antibodies

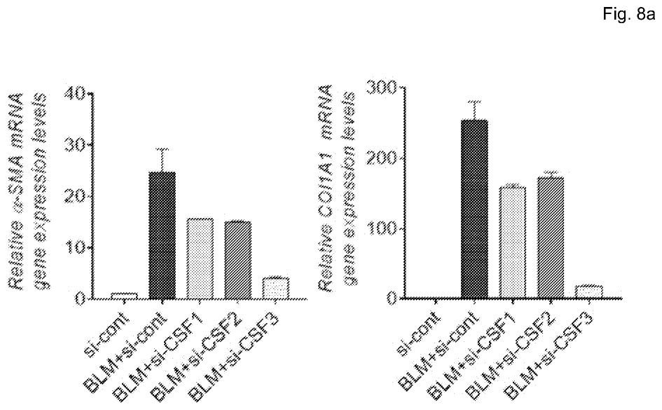

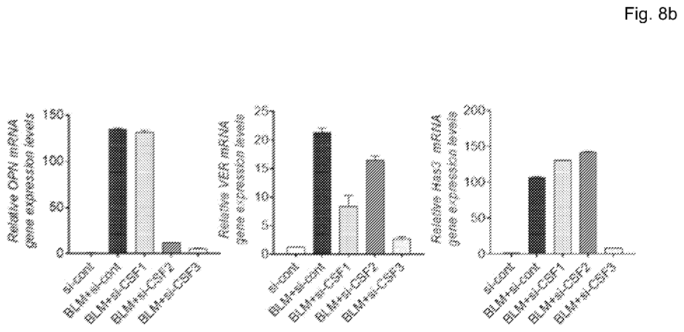

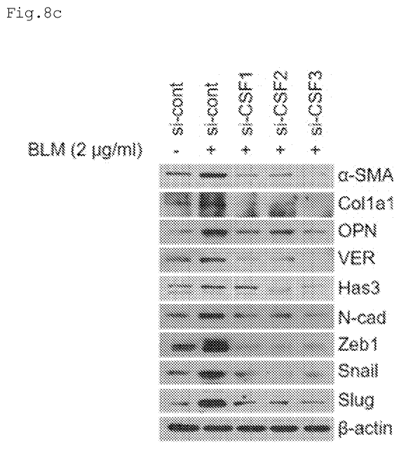

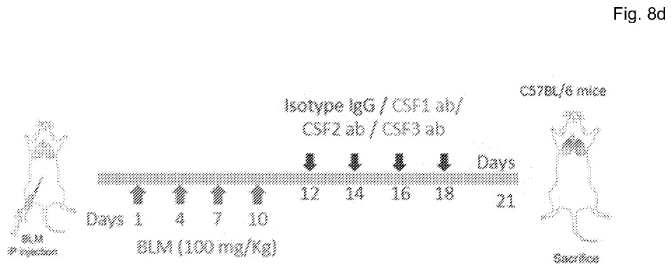

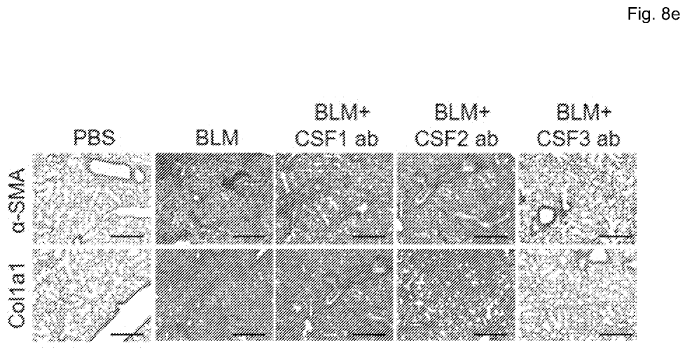

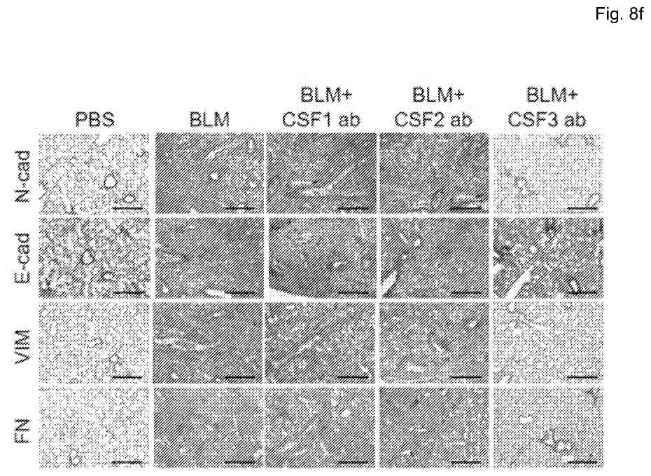

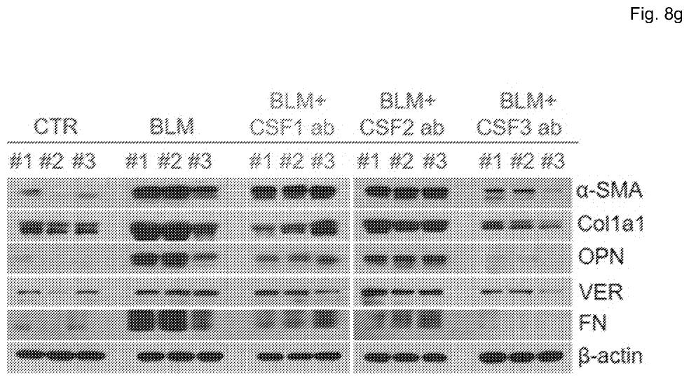

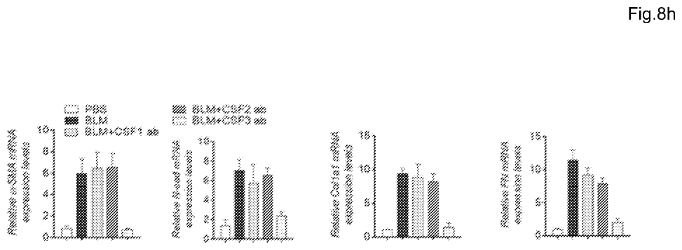

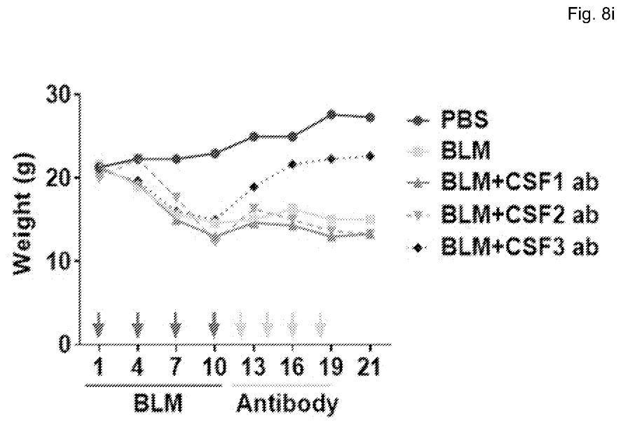

In addition to the anti-CSF3 neutralizing antibody, whose therapeutic effect was confirmed in Example 6-1, an experiment was conducted to compare the therapeutic effects of CSF family, CSF1, CSF2, and CSF3, on pulmonary fibrosis. When Beas-2b cells, in which pulmonary fibrosis was induced by bleomycin, were treated with the anti-CSF3 neutralizing antibody, it was confirmed that the expression of pulmonary fibrosis markers was inhibited. However, when treated with an anti-CSF1 neutralizing antibody or anti-CSF2 neutralizing antibody capable of inhibiting the activity of CSF1 or CSF2, respectively, no such change in expression was observed (FIGS. 8A to 8C). In the BLM-induced idiopathic pulmonary fibrosis mouse model, improvement in pulmonary fibrosis was confirmed only when the anti-CSF3 neutralizing antibody was treated, similar to the above in vitro cell experiment results. In addition, when treated with the anti-CSF1 neutralizing antibody or anti-CSF2 neutralizing antibody, a significant improvement effect was not confirmed (FIGS. 8D to 8H).

In addition, it was confirmed that one of the representative side effects of bleomycin, weight loss, was improved only in the mouse group treated with the anti-CSF3 neutralizing antibody (FIG. 8I).

Through the above results, the inventors of the present disclosure confirmed that among the CSF1, CSF2, and CSF3 belonging to the CSF family, the neutralizing antibody against CSF3 specifically exhibited the effect of treating pulmonary fibrosis.

### Example 7. Identification of Mechanism for Treating Pulmonary Fibrosis with CSF3-Neutralizing Antibody Treatment

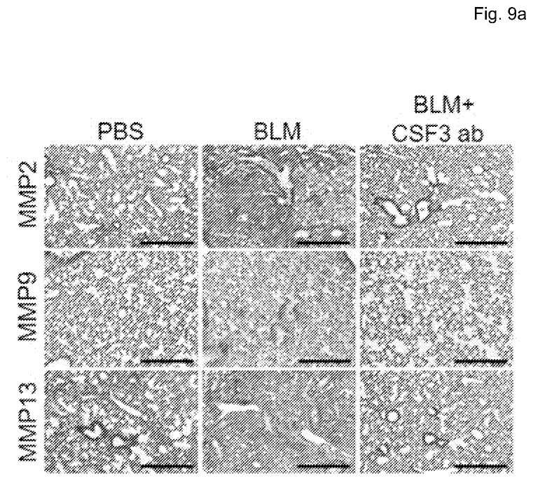

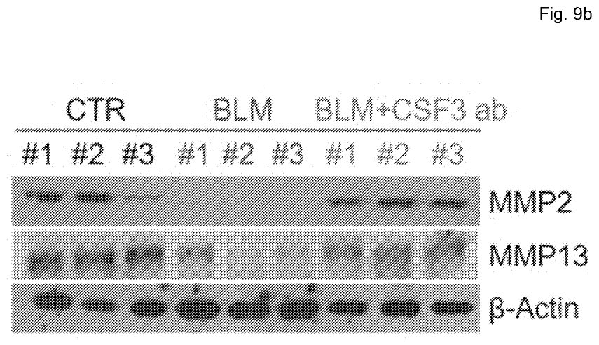

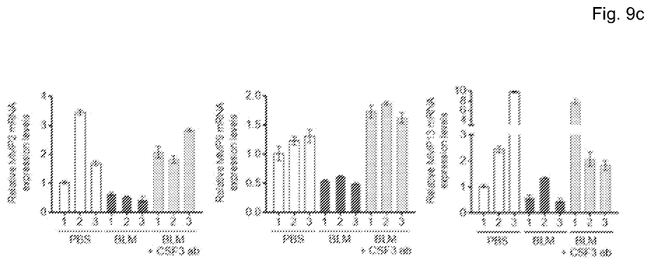

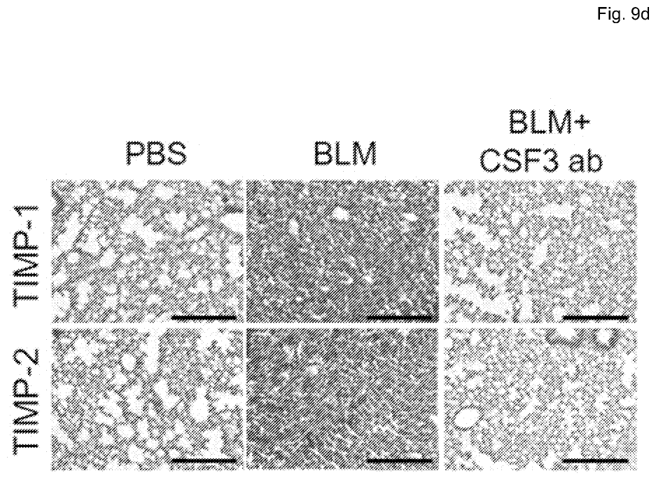

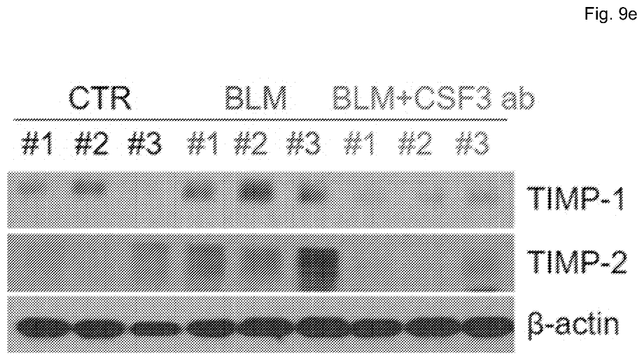

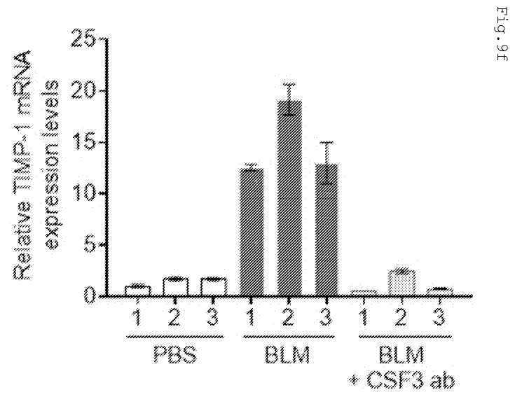

To identify a specific mechanism in which pulmonary fibrosis is improved by the anti-CSF3 neutralizing antibody identified in Example 6, the expression of matrix metalloproteinase (MMP) related to Extracellular matrix (ECM) degradation was analyzed in a BLM-induced idiopathic pulmonary fibrosis mouse model. Specifically, even though the expression of MMP2, MMP9, and MMP13 was decreased in the BLM-induced idiopathic pulmonary fibrosis mouse model, it was confirmed that the expression of MMP2, MMP9, and MMP13 was significantly increased in a group treated with the anti-CSF3 neutralizing antibody (FIGS. 9A to 9C).

In addition, when confirming the expression of tissue inhibitors of metalloproteinase (TIMP) serving as an inhibitor of the MMP, even though the expression of TIMP-1 and TIMP-2 was increased in the BLM-induced idiopathic pulmonary fibrosis mouse model, it was confirmed that the expression levels of TIMP-1 and TIMP-2 in the group treated with the anti-CSF3 neutralizing antibody was decreased to the level of a normal control group.

Through the above results, the inventors of the present disclosure confirmed that the anti-CSF3 neutralizing antibody of the present disclosure was able to treat pulmonary fibrosis by regulating the expression of MMP and TIMP.

### Example 8. Comparison of Therapeutic Effects of Conventional Therapeutic Agents and CSF3 Neutralizing Antibody on Pulmonary Fibrosis

8-1. Comparison of Therapeutic Effects of TGF-62 Antibody and Metformin

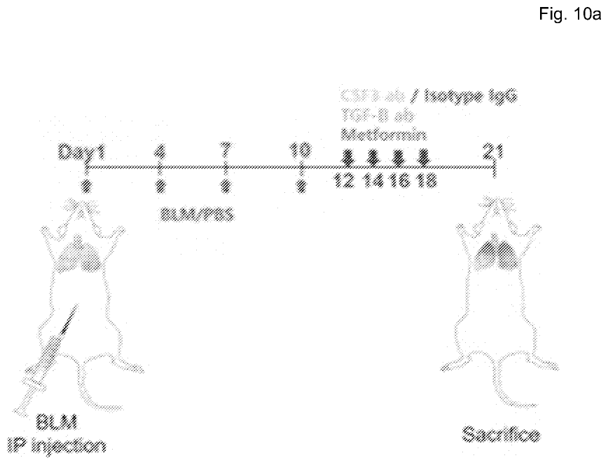

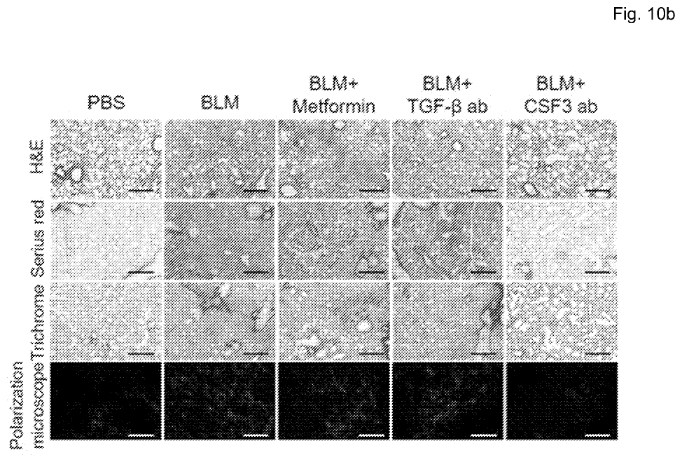

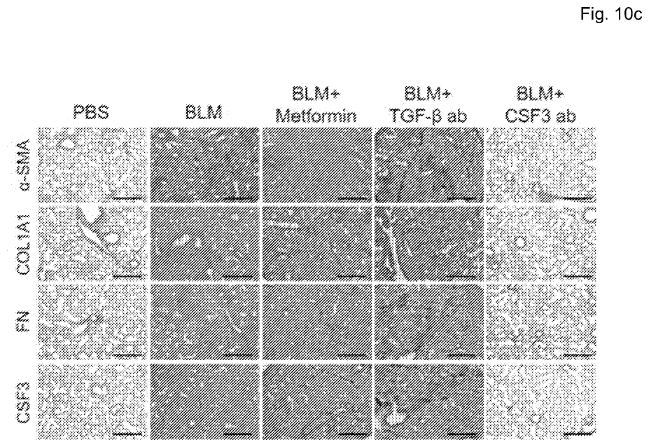

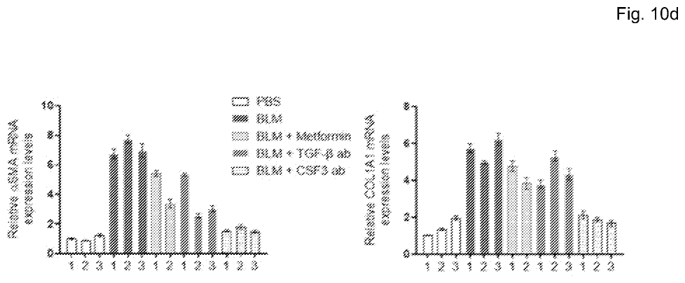

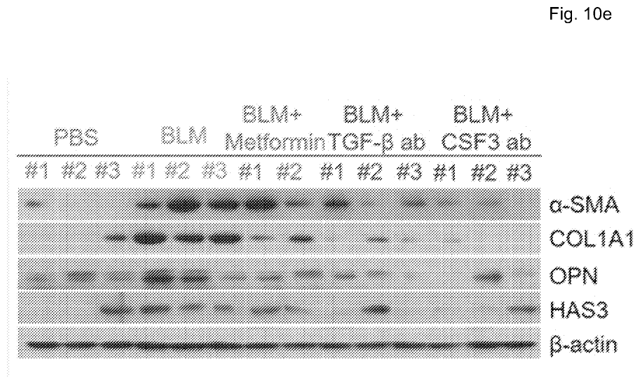

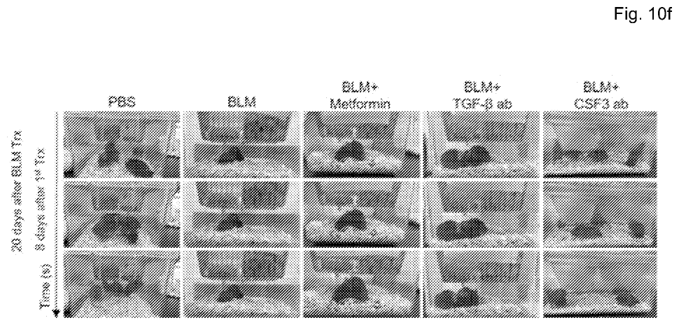

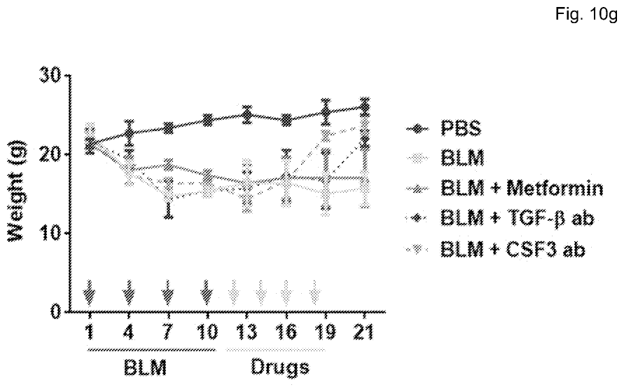

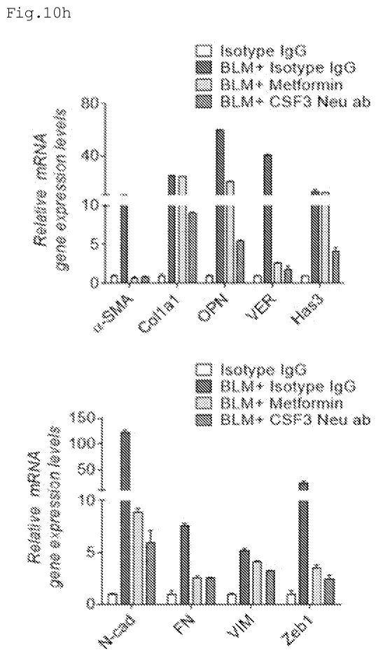

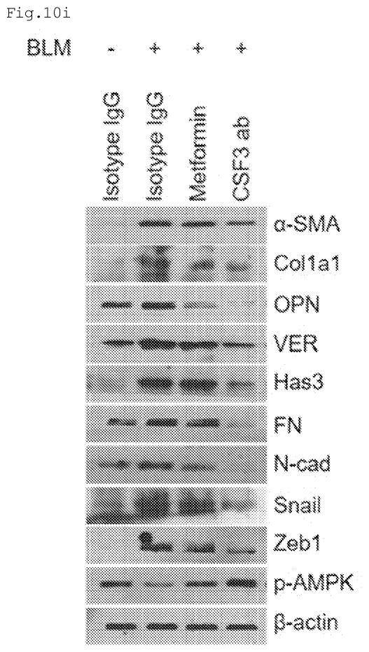

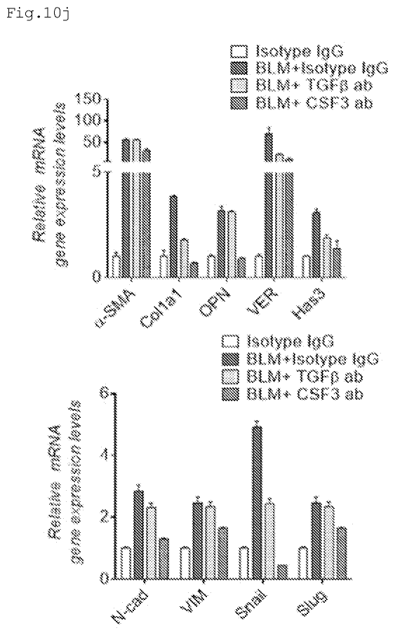

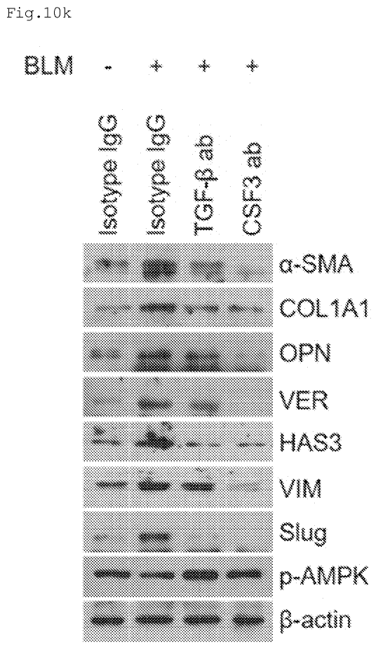

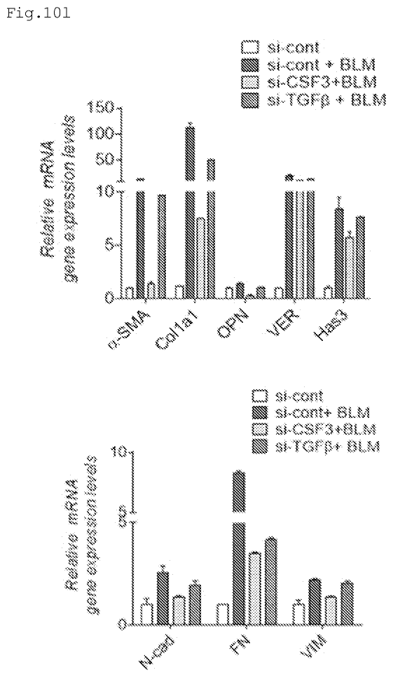

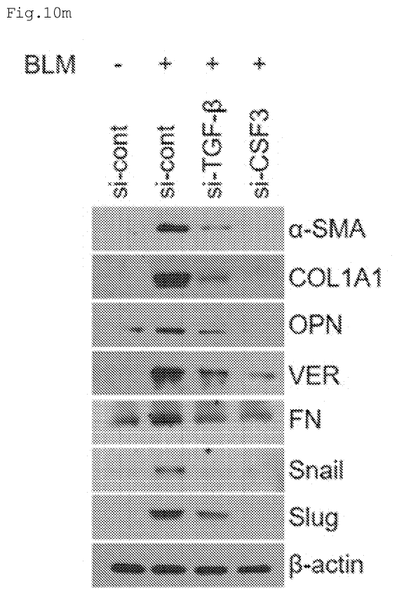

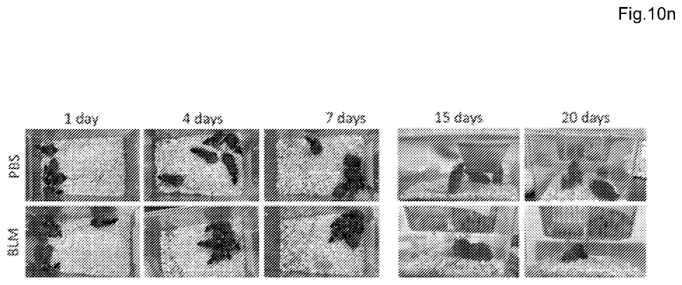

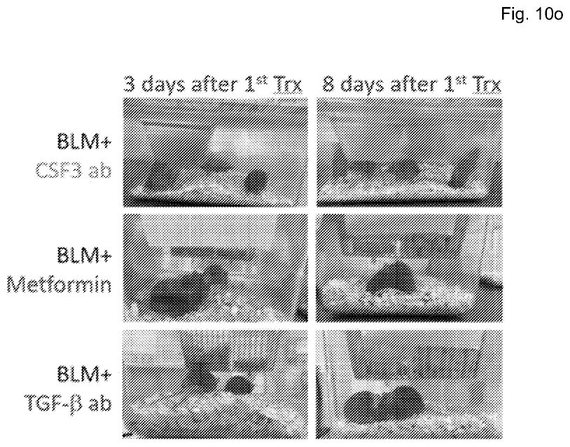

A comparison experiment was conducted to compare the improvement effect on pulmonary fibrosis through CSF3 inhibition confirmed specifically in Example 6 and the improvement effect of conventionally reported therapeutic target and substance (FIG. 10A). A BLM-induced idiopathic pulmonary fibrosis mouse model was treated with metformin, a TGF-β neutralizing antibody, or an anti-CSF3 neutralizing antibody while H&E staining, Sirius red staining, and trichrome staining were performed to observe the effects (FIG. 10B). As a result, it was confirmed that there was nearly little difference in the case of treating metformin treatment, a slight difference in the case of treating the TGF-S neutralizing antibody, and a significant difference in the case of treating anti-CSF3 neutralizing antibody of the present disclosure, from an untreated group. In addition, as a result of confirming the expression of pulmonary fibrosis markers and EMT markers by immunohistochemical staining (FIG. RT-qPCR (FIG. 10D), and Western blotting (FIG. 10E), it was confirmed that the expression of the pulmonary fibrosis markers and the EMT markers in a group treated with the anti-CSF3 neutralizing antibody was more significantly inhibited than that in other groups.

In addition, when treating the BLM-induced idiopathic pulmonary fibrosis mouse model with metformin, the TGF-β neutralizing antibody, or the anti-CSF3 neutralizing antibody, and then observing mouse behaviors over time (FIG. 10F), the BLM-induced idiopathic pulmonary fibrosis mouse model and a metformin-treated group had little movement. A slight movement was observed in a group treated with the TGF-β neutralizing antibody, and the group treated with the anti-CSF3-neutralizing antibody showed a movement similar to that of the control group. In addition, it was confirmed that one of the side effects induced by bleomycin, weight loss, was also improved to the most significant level when treated with the anti-CSF3 neutralizing antibody (FIG. 10G).

A bleomycin-treated lung epithelial cell line was treated with metformin, the TGF-β neutralizing antibody, or the anti-CSF3 neutralizing antibody while the expression levels of the pulmonary fibrosis markers and the EMT markers were confirmed. When treated with the anti-CSF3 neutralizing antibody, it was confirmed that the expression levels of the pulmonary fibrosis markers and the EMT markers were decreased. However, in a metformin-treated sample, no significant decrease was confirmed. When treated with the TGF-β neutralizing antibody, it was confirmed that the expression of the pulmonary fibrosis markers and the EMT markers was reduced, but the effect thereof was minor compared to the case of the anti-CSF3 neutralizing antibody (FIGS. 10J and 10K). Even when the expression of metformin, TGF-β, or CSF3 was inhibited by siRNA, not a neutralizing antibody, the expression of the pulmonary fibrosis markers and the EMT markers was more significantly inhibited in the case where CSF3 expression was inhibited than in other cases (FIGS. 10L and 10M).

To confirm the improvement effect over time, the BLM-induced idiopathic pulmonary fibrosis mouse model was raised for twenty days while the behaviors thereof were observed (FIG. 10N). The bleomycin-treated mice had significantly less movement than a control group without being treated with bleomycin when four days elapsed, and all of them died when twenty days elapsed. When treating the BLM-induced idiopathic pulmonary fibrosis mouse model with the anti-CSF3 neutralizing antibody, metformin, or the TGF-β neutralizing antibody, it was confirmed that most mice of a group treated with metformin or the TGF-β antibody did not move and died when eight days elapsed. However, it was confirmed that the group treated with the anti-CSF3 neutralizing antibody had an active movement similar to the level of the control group, even when eight days elapsed.

8-2. Comparison of Therapeutic Effect with Pirfenidone

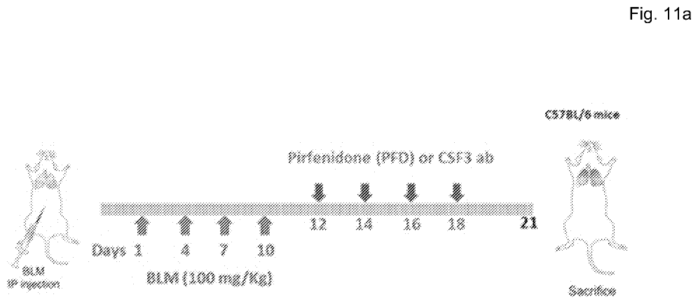

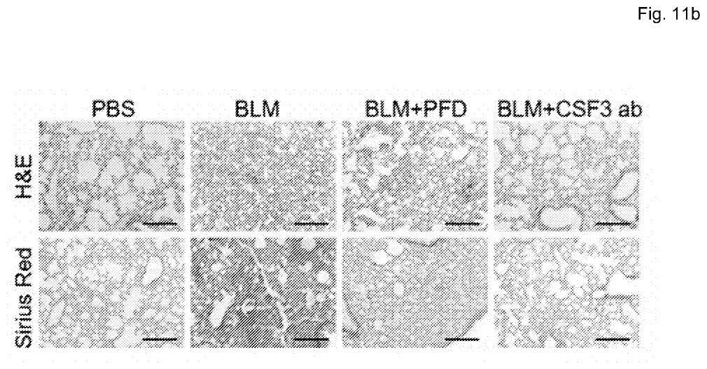

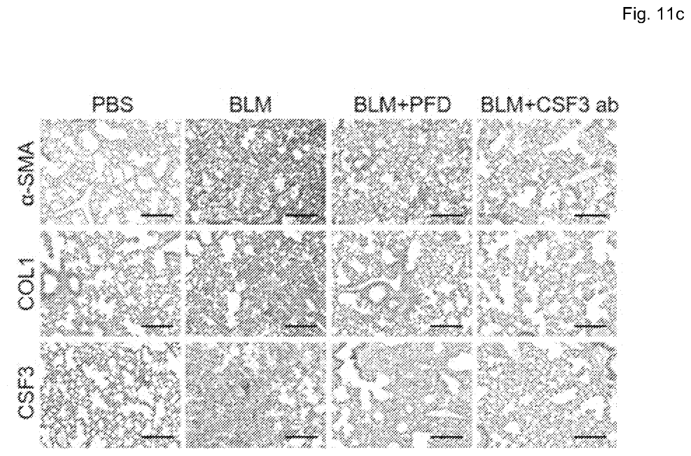

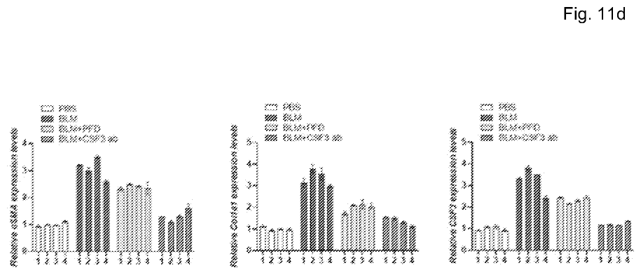

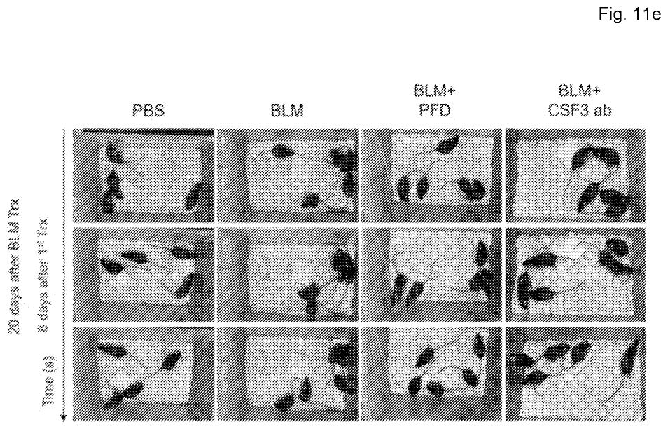

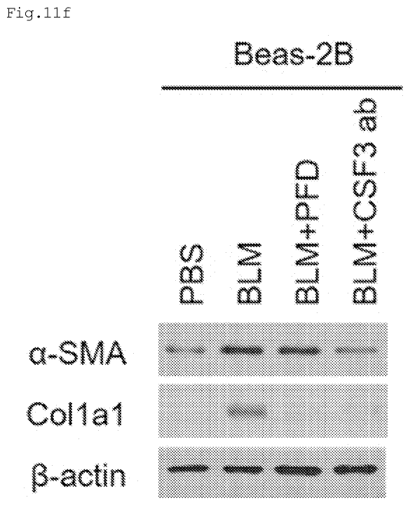

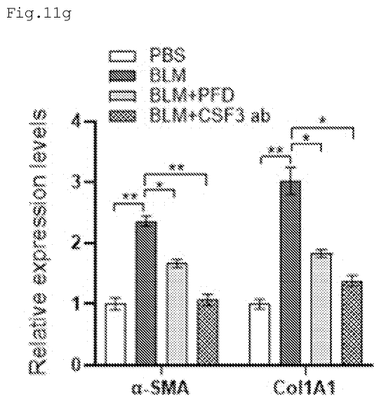

In order to compare the effect of improving pulmonary fibrosis with pirfenidone, an FDA-approved pulmonary fibrosis treatment conventionally used in clinical practice, pirfenidone and the anti-CSF3 neutralizing antibody were independently administered to the BLM-induced pulmonary fibrosis mouse model (FIG. 11A). As a result of performing H&E staining and Sirius red staining (FIG. 11B) after the administration, it was confirmed that a pirfenidone-treated group had a minor therapeutic effect compared to the BLM-induced pulmonary fibrosis mouse model, but a significant effect of improving pulmonary fibrosis was able to be observed in an experimental group treated with the anti-CSF3 neutralizing antibody. Even when performing immunohistochemical staining (FIG. 11C) and RT-qPCR (FIG. 11D) to confirm pulmonary fibrosis markers, it was confirmed that the experimental group treated with the anti-CSF3 neutralizing antibody obtained a significant inhibitory effect.

When observing the behaviors of mouse models, it was confirmed that the BLM-induced pulmonary fibrosis mouse model showed little movement, the group treated with pirfenidone showed a slight movement, and the group treated with the anti-CSF3 neutralizing antibody showed an active movement (FIG. 11E).

To validate the above results at a cellular level, Beas-2B cells were treated with bleomycin, and then experimental groups to which pirfenidone and the anti-CSF3 neutralizing antibody were each independently administered were prepared. Even when performing Western blotting (FIG. 11F) and RT-qPCR (FIG. 11G) to confirm the expression of the pulmonary fibrosis markers, it was confirmed that the expression was significantly decreased in the group treated with anti-CSF3 neutralizing antibody compared to the pirfenidone-treated group.

Through the above results, the inventors of the present disclosure confirmed that the anti-CSF3 neutralizing antibody had a significant effect of treating pulmonary fibrosis compared to pirfenidone, a pulmonary fibrosis drug currently used in clinical practice.

The above description of the present disclosure is given by way of illustration only, and it should be understood by those skilled in the art to which the present disclosure belongs that various changes and modifications can be made without departing from the technical spirit and scope of the present disclosure. Therefore, preferred embodiments of the present disclosure have been described for illustrative purposes, and should not be construed as being restrictive.

### INDUSTRIAL APPLICABILITY

When inhibiting CSF3, whose expression was increased in idiopathic pulmonary fibrosis induced by bleomycin, which is an anticancer agent, α-smooth muscle actin, collagen, and markers of epithelial-mesenchymal transition (EMT) were confirmed to be decreased. Developing CSF3-targeting drugs can treat pulmonary fibrosis by reducing epithelial-mesenchymal transition and accumulation of extracellular matrix components in lung epithelial cells of idiopathic pulmonary fibrosis patients or anticancer drug-induced idiopathic pulmonary fibrosis patients. Thus, the CSF3-targeting drugs are expected to be widely used in therapy fields of idiopathic pulmonary fibrosis.

1. A composition for combined anticancer therapy, comprising a Granulocyte-colony stimulating factor 3 (CSF3) inhibitor as an active ingredient.

2. The composition of claim 1,
wherein the composition is capable of inhibiting a side effect of an anticancer drug.

3. The composition of claim 2,
wherein the anticancer drug is a bleomycin.

4. The composition of claim 2,
wherein the side effect of the anticancer drug is an Idiopathic pulmonary fibrosis (IPF).

5. The composition of claim 1,
wherein the CSF3 inhibitor is an CSF3 antibody or anti-CSF3 siRNA.

6. The composition of claim 1,
wherein the composition is capable of inhibiting a differentiation of lung cells to myofibroblasts.

7. The composition of claim 1,
wherein the composition is capable of inhibiting an Epithelial to Mesenchymal Transition (EMT).

8. The composition of claim 1,
wherein the composition is capable of inhibiting an Extra Cellular Matrix remodeling (ECM remodeling).

9. The composition of claim 6,
wherein the inhibition of the differentiation into myofibroblasts is induced by an inhibition of a α-Smooth Muscle Actin (α-SMA).

10. The composition of claim 7,
wherein the inhibition of the EMT is induced by an inhibition of one or more protein selected from a group consisting of Fibronectin (FN), Vimentin (VIM), and ZEB1.

11. The composition of claim 7,
wherein the inhibition of the EMT is induced by an inhibition of a STATS protein.

12. The composition of claim 8,
wherein the inhibition of ECM remodeling is induced by an inhibition of one or more protein selected from a group consisting of Versican, Osteopontin (OPN), Collagen, and HAS3.

13. The composition of claim 8,
wherein the inhibition of ECM remodeling is induced by an increase of a matrix metalloproteinase (MMP) protein.

14. The composition of claim 8,
wherein the inhibition of ECM remodeling is induced by a decrease of a tissue inhibitors of metalloproteinase (TIMP) protein.

15. The composition of claim 1,
wherein the composition is characterized in that it is administered simultaneously or sequentially with an anticancer drug.

16. A combination agent for anticancer, comprising an anticancer drug and the composition of claim 1.

17. A pharmaceutical composition for preventing or treating a pulmonary fibrosis disease, comprising a Granulocyte-colony stimulating factor 3 (CSF3) inhibitor as an active ingredient.

18. The pharmaceutical composition of claim 17,
wherein the pulmonary fibrosis disease is induced by an anticancer drug.

19. The pharmaceutical composition of claim 2,
wherein the anticancer drug is a bleomycin.

20. The pharmaceutical composition of claim 17,
wherein the pulmonary fibrosis disease comprises a myofibroblast hyperplasia of pulmonary cells or Idiopathic pulmonary fibrosis (IPF).

21. The pharmaceutical composition of claim 17,
wherein the CSF3 inhibitor is an anti-CSF3 antibody or anti-CSF3 siRNA.

22. The pharmaceutical composition of claim 17,
wherein the pharmaceutical composition is capable of inhibiting a differentiation of lung cells into myofibroblasts.

23. The pharmaceutical composition of claim 17,
wherein the pharmaceutical composition is capable of inhibiting an Epithelial to Mesenchymal Transition (EMT).

24. The pharmaceutical composition of claim 17,
wherein the pharmaceutical composition is capable of inhibiting an Extra Cellular Matrix remodeling (ECM remodeling).

25. The pharmaceutical composition of claim 22, wherein the inhibition of the differentiation into myofibroblasts is induced by an inhibition of a α-Smooth Muscle Actin (α-SMA).

26. The pharmaceutical composition of claim 23,
wherein the inhibition of the EMT is induced by an inhibition of one or more protein selected from a group consisting of Fibronectin (FN), Vimentin (VIM), and ZEB1.

27. The pharmaceutical composition of claim 23,
wherein the inhibition of the EMT is induced by an inhibition of a STAT3 protein.

28. The pharmaceutical composition of claim 24,
wherein the inhibition of ECM remodeling is induced by an inhibition of one or more protein selected from a group consisting of Versican, Osteopontin (OPN), Collagen, and HAS3.

29. The pharmaceutical composition of claim 24,
wherein the inhibition of ECM remodeling is induced by an increase of a matrix metalloproteinase (MMP) protein.

30. The pharmaceutical composition of claim 24,
wherein the inhibition of ECM remodeling is induced by a decrease of a tissue inhibitors of metalloproteinase (TIMP) protein.

31. A method for treating a pulmonary fibrosis disease, comprising:
administering the pharmaceutical composition of claim 17 into a subject.

32. The pharmaceutical composition of claim 17 for use in treating a pulmonary fibrosis disease.
